(19) United States
(12) Patent Application Publication
(10) Pub. No.: US 2012/0225123 A1
(43) Pub. Date: Sep. 6, 2012

(54) Use of Synergistic Combinations of an Avermectin and an Antineoplastic Compounds for the Treatment of Hematological Malignancies

(75) Inventors: Aaron David Schimmer, Thornhill (CA); Sumaiya Sharmeen, Tornhill (CA); Marko Skrtic, Toronto (CA)

(73) Assignee: UNIVERSITY HEALTH NETWORK, Toronto, ON (CA)

(21) Appl. No.: 13/504,578

(22) PCT Filed: Nov. 9, 2010
(86) PCT No.: PCT/CA2010/001780

§ 371 Date: May 25, 2012

Related U.S. Application Data
(60) Provisional application No. 61/259,395, filed on Nov. 9, 2009.

(51) Int. Cl. A61K 31/7048; A61K 9/28; A61P 35/02; A61P 35/00; A61K 31/7068

(52) U.S. Cl. 424/474; 514/30

(57) ABSTRACT

The disclosure relates to a method of treating a hematological malignancy comprising administering to a subject in need thereof a synergistic combination of a first compound comprising an effective amount of one or more Avermectins and a second compound comprising a chemotherapeutic, preferably daunorubicin, cyarabine, doxorubicin, idarubicin mitoxantrone, amsacrine, and mixtures thereof.

### RELATED APPLICATIONS

This is a Patent Cooperation Treaty Application which claims the benefit of 35 U.S.C. 119 based on the priority of corresponding U.S. Provisional Patent Application No. 61/259,395 filed Nov. 9, 2009, which is incorporated herein in its entirety.

### FIELD OF THE DISCLOSURE

The disclosure relates to methods and compositions for the treatment of hematological malignancies and particularly to combination compositions and therapies for the treatment of hematological malignancies such as acute myeloid leukemia (AML) or acute lymphoid leukemia (ALL) in a subject.

### BACKGROUND OF THE DISCLOSURE

Ivermectin (IVM) is a derivative of avermectin B1 and licensed for the treatment of strongyloidiasis and onchocerciasis parasitic infections and other worm infestations (e.g., ascariasis, trichuriasis and enterobiasis). As part of the development of this agent as an antiparasitic agent, IVM was extensively evaluated for its pharmacology, safety and toxicity in humans and animals. For example, the LD₅₀ of oral IVM in mice, rats and rabbits ranges from 10 to 50 mg/kg⁷. In humans, when used to treat onchocerciasis, 100-200 μg/kg of IVM is administered as a single dose⁸. This brief and low-dose treatment is sufficient to achieve an anti-parasitic effect, but higher doses and treatment beyond one day have been safely administered for other conditions. For example, in patients with spinal injury and resultant muscle spasticity, up to 1.6 mg/kg of IVM was administered subcutaneously at twice weekly for up to 12 weeks. In this study, no significant adverse effects were reported.⁹ Likewise, to evaluate the safety of oral IVM, healthy volunteers received 30-120 mg on days 1, 4 and 7 and then a further dose in week 3.¹⁰ Even at a dose of 120 (˜2 mg/kg) no serious adverse effects were noted. Finally, reports of IVM overdoses also support the evaluation of high doses of IVM in humans, as in the majority of these cases, no serious adverse events were reported.¹¹

### SUMMARY OF THE DISCLOSURE

An aspect of the disclosure includes a method of treating a hematological malignancy comprising administering to a subject in need thereof, an effective amount of an avermectin, in combination with an effective amount of a chemotherapeutic.

Another aspect of the disclosure includes use of an effective amount of an avermectin, in combination with an effective amount of a chemotherapeutic for the treatment of a hematological malignancy.

A further aspect of the disclosure includes use of an avermectin, in combination with an effective amount of a chemotherapeutic for the manufacture of a medicament for the treatment of a hematological malignancy.

A further aspect includes a method of inducing cell death in a hematological cancer cell comprising contacting the cell with an avermectin and a chemotherapeutic.

Yet a further aspect of the disclosure includes an avermectin, in combination with an effective amount of a chemotherapeutic or the treatment of a hematological malignancy.

A further aspect of the disclosure includes a composition compromising an avermectin, in combination with an effective amount of a chemotherapeutic for the treatment of a hematological malignancy.

Another aspect of the disclosure includes a composition comprising an avermectin, in combination with cytarabine and/or daunorubicin.

Also included in other aspects of the disclosure are kits comprising an avermectin and instructions for administering in combination with and cytarabine and/or daunorubicin.

A further aspect includes a pharmaceutical pack comprising a composition disclosed herein and optionally instructions for use.

In an embodiment, the chemotherapeutic is selected from cytarabine, daunorubicin, doxorubicin, idarubicin, mitoxantrone, and amsacrine and mixtures thereof.

In a further embodiment, the chemotherapeutic is cytarabine or daunorubicin.

In an embodiment, the avermectin is selected from ivermectin (IVM), invermectin, avermectin, abamectin, doramectin, eprinomectin and selamectin and mixtures thereof.

In a further embodiment, the avermectin is IVM.

In an embodiment, the hematological malignancy is a leukemia, myeloma or lymphoma and/or a relapsed or refractory hematological malignancy. In a further embodiment, the leukemia is selected from acute myeloid leukemia (AML), acute lymphocytic leukemia (ALL) and chronic myelogenous leukemia (CML).

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

FIG. 1: A screen of off-patent drugs identifies antiparasitic agent ivermectin that reduces viability of leukemia cells in vitro and reduces clonogenic growth of leukemia cells when incubated with methylcellulose for seven days.

(A) A high throughput screen with a small chemical library (n=100) focused on anti-microbials and metabolic regulators with wide therapeutic windows and well understood pharmacokinetics identified ivermectin as a potential anti-cancer agent. OCI-AML2 cells were incubated with aliquots of this chemical library at five concentrations (3-50 μM) and viability was measured using MTS assay after 72 hours as described in the Methods and Materials section. Data represent the percentage of viable OCI-AML2 cells (y axis) and the compounds (6 μM) sorted in increasing potency (x axis). B) Leukemia cell lines were treated with increasing concentrations of ivermectin. Seventy-two hours after incubation, cell growth and viability was measured by the MTS assay. Data represent the mean EC50 and 95% Cl from 3 independent experiments. C) Primary normal hematopoietic cells (PBSC) (n=3), primary AML patient samples (AML) (n=3) and U937 leukemia cells were treated with increasing concentrations of IVM for 48 hours. After incubation, cell viability was measured by Annexin V and PI staining. Data represent the mean±SD percent viable cells from experiments performed in triplicate. D) Primary AML cell samples (AML) (n=6) and normal hematopoietic blood stem cell samples (PBSC) (n=3) were treated with ivermectin (6 μM) for 24 hours and then plated in a methylcellulose colony forming assay. Colonies were counted seven days (AML samples) or 14 days (normal PBSC) after plating. Data represent the mean±SD percent colony formation compared to control treated cells. E) OCI AML2 cells were treated with increasing concentrations of ivermectin with or without the pan-caspase inhibitor, Zvad (50 μM). After 24 hours of incubation, cell death was assessed by Annexin V-PI staining. Data represent the mean±SD percent viable cells from experiments performed in triplicate. F) OCI AML2 cells were treated with 3 μM ivermectin or buffer control overnight. After treatment, cells were stained with PI and the DNA content was measured by flow cytometry. A representative figure is shown. G) Primary AML cells were plated directly into MethoCult GF H4434 medium containing ivermectin (3 and 6 μM). Seven days after plating, the number of colonies was counted. Data represent the mean±SD percent colony formation compared to control treated cells (n=3).

FIG. 2: Ivermectin delays tumor growth, reduces tumor weight in leukemia mouse xenografts and leads to apoptosis in vivo

Sublethally irradiated NOD/SCID mice were injected subcutaneously with K562 cells (n=20; 10 per group) (A,B), OCI-AML2 human leukemia cells (n=20; 10 per group) (C,D) or MDAY-D2 murine leukemia cells (n=20; 10 per group) (E,F). After implantation, mice were treated with ivermectin daily for 10 days (K562) or treated with 8 doses over 10 days (OCI-AML2) with IVM (3 mg/kg) by oral gavage in water or vehicle control. MDAY-D2 mice were treated similarly but dosage escalated from 3 mg/kg (4days) to 5 mg/kg (3 days) and 6 mg/kg (3 days) as the drug was well tolerated. Fourteen (MDAY-D2), 15 (OCI-AML2) or 17 (K562) days after injection of cells, mice were sacrificed, tumors excised and the volume and weight of the tumors were measured. The tumor weight and the mean volume±SEM are shown. Differences in tumor volume and weight were analyzed by an unpaired t-test: ***p<0.0001; **p<0.001; *p<0.05. G) OCI-AML2 cells (2.5×10⁵) were injected subcutaneously into the flanks of sub-lethally irradiated NOD/SCID mice. Once tumors were established, mice were treated with ivermectin (7 mg/kg) or vehicle control intraperitoneally for 5 days. After treatment, mice were sacrificed and tumors were harvested. Evidence of apoptosis was measured by Tunel staining and immunohistochemistry. The stained samples were scanned using Aperio Scanscope XT at 20× magnification, which gives a resolution of 0.5 μm/pixel and analyzed using Aperio ImageScope. A representative section from the tumors of control and ivermectin-treated mice are shown.

FIG. 3: Ivermectin induces chloride influx and increases cell size in leukemia cells

A) OCI-AML2 leukemia and DU145 prostate cancer cells were treated with increasing concentrations of IVM. After 24 hours of incubation, cell viability was measured by Annexin V and PI staining. Data represent the mean+SD percent viable cells. B) OCI-AML2 and C) DU145 cells were treated with 10 pM IVM for 2 hours and levels of intracellular chloride were measured by staining cells with the fluorescent dye SPQ that is quenched upon binding chloride. Histograms from representative experiments are shown. D) OCI-AML2 and E) DU145 cells were treated with 6 and 10 μM ivermectin for 2 hours. After treatment, cell size was measured by forward light scatter and flow cytometry. Data represent mean±SD fold change in cell size compared to control from representative experiments performed in triplicate. **p<0.01, by unpaired t-test.

FIG. 4: Ivermectin induces plasma membrane hyperpolarization dependent on chloride influx OCI-AML2 cells were treated with increasing concentrations of IVM for 24 hours (A) or 6 μM of IVM for increasing times of incubation (B). After treatment, plasma membrane potential was measured by staining cells with DiBAC4(3) and flow cytometry. Data represent the mean±SD fold change in plasma membrane potential compared to control treated cells. Representative experiments performed in triplicate are shown. Differences in fold change of membrane potential compared to control were analyzed by an unpaired t-test: ***p<0.0001; *p<0.05. U937 and TEX leukemia cells, a primary AML sample (AML) (C), DU145 and PPC-1 prostate cancer, and two samples of normal hematopoietic cells (D), were treated with 6 μM of ivermectin for increasing times. After treatment, plasma membrane potential was measured as above. Data represent the mean±SD fold change in plasma membrane potential compared to control treated cells. Representative experiments performed in triplicate are shown. Differences in change of membrane potential compared to control were analyzed by an unpaired t-test: ***p<0.001; *p<0.05. (E) OCI-AML2 cells were treated with 6 μM IVM in chloride replete and chloride free media for 5 hours. After incubation, plasma membrane hyperpolarization was measured as above. Data represent the mean±SD plasma membrane potential compared to untreated cells in chloride-replete media. Representative experiments performed in triplicate are shown. Differences in membrane potential compared to control were analyzed by an unpaired t-test: ***p<0.0001; *p<0.05. OCI-AML 2 cells were treated with ivermectin (6 μM) (F). Five hours after treatment, cytosolic calcium concentration was detected by staining cells with the fluorescent dye, Indo-1 AM and flow cytometry analysis. Representative histograms are shown. As a control for cytosolic calcium influx, OCI-AML2 cells were treated with digoxin (25 nM) for 5 hours (G). Representative histograms are shown.

FIG. 5: Ivermectin induces generation of reactive oxygen species. OCI-AML 2 leukemia cells were treated with increasing concentrations of IVM for over night (A) or 6 μM of IVM for increasing times of incubation (B). After incubation, intracellular Reactive oxygen (ROS) species were detected by staining cells with Carboxy-H₂DCFDA (final concentration 10 μM) and flow cytometric analysis. Data represent the mean±SD fold change in ROS production compared to control. Representative experiments performed in triplicate are shown. Differences in change of ROS compared to control were analyzed by an unpaired t-test: ***p<0.001; **p<0.005. (C) U937 and TEX leukemia cells, DU145 and PPC-1 prostate cells were treated with ivermectin at 6 μM for 2 hours. After treatment, ROS generation was measured as above. Data represent the mean±SD fold change in ROS production compared to each of their buffer treated controls. Representative experiments performed in triplicate are shown. Differences in change of ROS compared to control were analyzed by an unpaired t-test: ***p<0.001. Primary AML cells (n=3) and normal hematopoietic stem cells (PBSC, n=3) were treated with ivermectin (6 mM) for 6 hours. After treatment, ROS generation was measured as above. Data represent the mean±SD fold change in ROS production compared to each of their buffer treated controls for experiments performed in triplicate. Differences in ROS production compared to control were analyzed by an unpaired t-test: *** p<0.001. (D) OCI-AML2 cells were treated simultaneously with IVM (3 μM), the ROS scavenger, N-acetyl-L-Cysteine (NAC) (5 μM) or the combination of NAC with IVM. After 48 hours of treatment, cell growth and viability were measured by the MTS assay. Data represent the mean±SD percent viable cells from a representative experiment performed in triplicate. Differences in change of cell viability compared to control were analyzed by an unpaired t-test: ***p<0.001.

FIG. 6: Ivermectin increases expression of STAT1 and its target genes through a ROS dependent mechanism

(A) OCI-AML2 cells were treated with 3 μM ivermectin (IVM) for 30 hours. After treatment, RNA was isolated, reverse transcribed and subjected to quantitative PCR using specific primers for STAT1A, STAT1B and STAT1 target genes OAS1, TRIM22 and IFIT3. Data represent mean±SD fold increase in gene expression normalized to 18S expression and compared to control cells. (B) OCI AML2, 0937 and HL60 leukemia, and DU145 and PPC-1 prostate cancer cells were treated with 6 μM ivermectin for 24 hours and mRNA levels of STAT1A and STAT1B were measured using quantitative PCR and normalized to 18S expression as (A). Data represent mean±SD fold increase in gene expression compared to control cells. (C) OCI-AML2 cells (2.5×10⁵) were injected subcutaneously into the flanks of sub-lethally irradiated NOD/SCID mice. Once tumors were established, mice were treated with ivermectin (7 mg/kg) intraperitoneally or vehicle control for 5 days (n=3 per group). After treatment, mice were sacrificed, and tumors harvested. mRNA was extracted and changes in STAT1A and 1B expression were measured by Q-RT-PCR. Data represent mean±SD fold increase in gene expression normalized to 18S expression compared to tumors from control treated mice. (D) OCI-AML2 cells were treated simultaneously with ivermectin (3 μM), the ROS scavenger N-acetyl-L-cysteine (NAC) (5 μM), or both for 30 hours, and STATIA and STAT1B expression assessed as described for Panel A. Relative expression values normalized to 18S are reported as fold-change±SD compared to the untreated control for each gene.

FIG. 7: Ivermectin synergizes with cytarabine and daunorubicin to induce cell death in leukemia cells.

OCI-AML2 cells were treated with increasing concentrations of daunorubicin (A) and cytarabine (B) for overnight. After treatment, ROS production was measured by staining cells Carboxy-H₂DCFDA (final concentration 10 μM) and flow cytometric analysis. Data represent the mean±SD fold change in ROS production compared to control. Representative experiments performed in triplicate are shown. The effects of different concentrations of IVM in combination with cytarabine and daunorubicin on the viability of OCI-AML2 and U937 cells were measured by MTS assay after 72 hours of incubation. Data were analyzed with Calcusyn software by the Calcusyn median effect model. Combination index (CI) versus Fractional effect (Fa) plot showing the effect of the combination of IVM with daunorubicin (C) and IVM with cytarabine (D). CI<1 indicates synergism. Representative isobolograms of experiments performed in triplicate are shown. (E) Normal hematopoietic cells (PBSC) (n=2) were treated with T increasing concentrations of ivermectin and cytarabine (0, 2.5 and 5 μM). After 48 hours, cell viability was measured by Annexin V-PI staining. Data represent the mean±S percent of viable cells from experiments performed in triplicate. (F) OCI-AML2 (i) and U937 (ii) cells were treated with ivermectin, cytarabine or the combination of the two drugs at varying concentrations for 72 hours. Ivermectin→cytarabine denotes that ivermectin was added initially and cytarabine was added for the last 48 hours of the 72 hour experiment. Cytarabine→ivermectin denotes that cytarabine was added initially and ivermectin was added for the last 48 hours of the 72 hour experiment.

### DETAILED DESCRIPTION OF THE DISCLOSURE

I. Definitions

The term “avermectin” as used herein refers to a group of macrocyclic lactones produced by the bacterium Streptomyces avermitilis (Reynolds JEF (Ed) (1993) Martindale, The extra pharmacopoeia, 29th Edition, Pharmaceutical Press, London) comprising four closely-related major components, A1a, A2a, B1a and B2a, and four minor components, A1b, A2b, B1b and B2b as shown in Formula (I):

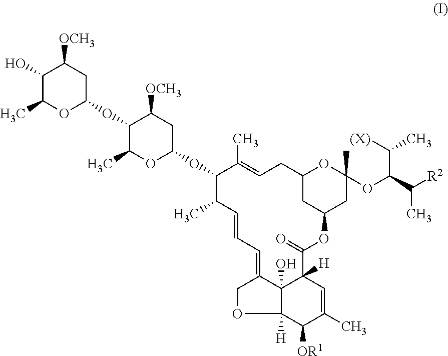

wherein the designations for the variable groups are as follows:

| Compound | X | R¹ | R² |
|---|---|---|---|
| A1a | —CH═CH— | CH₃ | C₂H₅ |
| A1b | —CH═CH— | CH₃ | CH₃ |
| A2a | —CH₂CH(OH)— | CH₃ | C₂H₅ |
| A2b | —CH₂CH(OH)— | CH₃ | CH₃ |
| B1a | —CH═CH— | H | C₂H₅ |
| B1b | —CH═CH— | H | CH₃ |
| B2a | —CH₂CH(OH)— | H | C₂H₅ |
| B2b | —CH₂CH(OH)— | H | CH₃ |

or a pharmaceutically acceptable solvate and/or prodrug thereof, or mixtures thereof. Avermectins can be synthesized, for example, isolated from natural sources, or semi-synthesized. (Avermectin aglycons. Helmut Mrozik, Philip Eskola, Byron H. Arison, George Albers-Schoenberg, Michael H. Fisher J. Org. Chem., 1982, 47 (3), pp 489-492; Ivermectin-derived leishmanicidal compounds, Falcäo C A, Muzitano M F, Kaiser C R, Rossi-Bergmann B, Férézou J P. Bioorg Med Chem. 2009 Jan. 15; 17(2):496-502). Avermectins include, in particular, ivermectin, invermectin, avermectin, abamectin, doramectin, eprinomectin and selamectin, and mixtures thereof and solvates and/or solvates thereof. Solvates of avermectins include, for example, those described in PCT patent application publication nos. WO 95/10525 and WO 99/07721. Avermectins also include known derivatives of avermectins such as the 5-oxime avermectins described in U.S. Pat. No. 5,015,630.

The term “avermectin that generates radical oxygen species in a leukemia cell” refers to an avermectin, which induces chloride influx induced ROS production sufficient to induce ROS dependent gene expression when a sufficient amount (e.g. comparable to IVM) is contacted with a leukemia cell, such as an AML cell. An avermectin that generates radical oxygen species in a leukemia cell includes for example, ivermectin, and can be determined using methods such as those described in the Examples.

The term “ivermectin” or “IVM” as used herein means a mixture of compounds of the Formula (II):

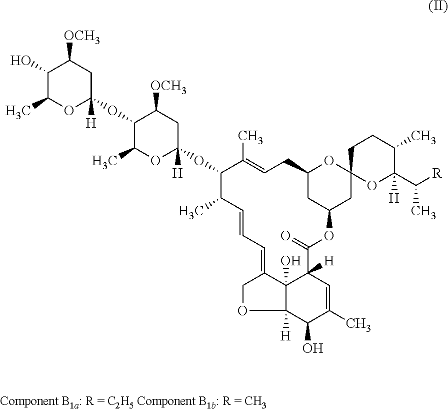

or a pharmaceutically acceptable solvate and/or prodrug thereof. IVM is a mixture of two compounds, namely 22,23-dihydroavermectin B₁ₐ and 22,23-dihydroavermectin B_{1b}, which are also referred to as 5-O-demethyl-22,23-dihydroavermectin A₁ₐ and 5-O-demethyl-22,23-dihydroavermectin A_{1b} or H₂B₁ₐ and H₂B_{1b} respectively. For example, ivermectin can contain at least 80%, for example about 90% of 22,23-dihydroavermectin B₁ₐ and less than 20%, for example about 10% of 22,23-dihydroavermectin B_{1b}. In another example, ivermectin can contain about 80% of 22,23-dihydroavermectin B₁ₐ and about 20% of 22,23-dihydroavermectin B_{1b}. IVM is sold for example, under the brand name Stromectol®.

The term “chemotherapeutic” or “antineoplastic agent” refers to compounds or combinations of compounds for treating cancer and includes for example alkylating agents, antimetabolites, anthracyclines, anthracenedione, plant alkaloids, and topoisomerase inhibitors, as well as proteasome inhibitors, demethylating agents, kinase inhibitors, microtubule poisons.

The term “chemotherapeutic DNA damaging drug” as used herein refers to the subset of chemotherapeutic drugs that interact with and/or modify DNA and include without limitation alkylating agents, and anthracyclines, and antimetabolites.

The term “anthracycline” as used herein refers to a class of drugs used in cancer chemotherapy derived from Streptomyces bacteria that damage DNA, and includes for example, daunorubicin, doxorubicin, idarubicin and epirubicin.

The term “hematological malignancy chemotherapeutic” as used herein means a compound for treating a hematological malignancy, for example AML or ALL, such as an anthracycline, or an anthracenedione (e.g. mitoxantrone), and in an embodiment means a compound selected from cytarabine, daunorubicin, doxorubicin, idarubicin mitoxantrone, and amsacrine and mixtures thereof.

The term “cytarabine”, also known as “AraC”, “aracytidine” and “cytosine arabinoside” means a compound having the structure:

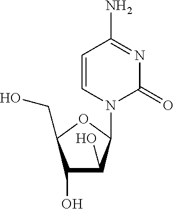

or a pharmaceutically acceptable salt, solvate, and/or prodrug thereof. Cytarabine is sold for example, under the brand names AR3, Alexan, Arabitin, Arafcyt, Cytarbel, Cytosar, Cytosar-U, Depocyt, Depocyt (liposomal), Erpalfa, Iretin, Spongocytidine, Tarabine, Ara-C and Udicil.

The term “daunorubicin” as used herein means a compound having the structure:

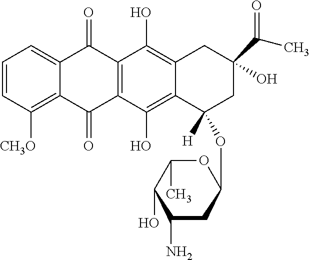

or a pharmaceutically acceptable salt, solvate and/or prodrug thereof. Acceptable salts include for example hydrochloride and citrate salts. Daunorubicin is for example sold under the brand names DaunoXome® (liposomal formulation) and Cerubidine® (daunorubicin hydrochloride formulation).

The term “doxorubicin” as used herein means a compound having the structure:

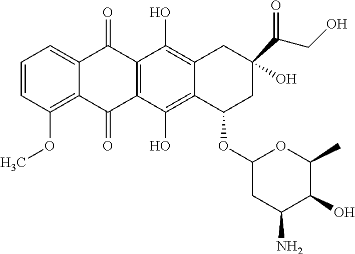

or a pharmaceutically acceptable salt, solvate and/or prodrug thereof. Acceptable salts include for example hydrochloride and citrate salts. Doxorubicin is sold for example, under the brand names Adriamycin, Adriamycin PFS, Adriamycin RDF, Adriblastin or Rubex.

The term “idarubicin” as used herein means a compound having the structure:

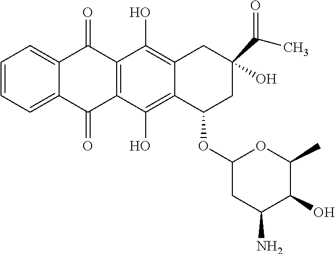

or a pharmaceutically acceptable salt, solvate and/or prodrug thereof as well as mixtures thereof.

The term “mitoxantrone” as used herein means a compound having the structure:

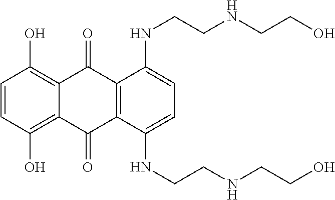

or a pharmaceutically acceptable salt, solvate and/or prodrug thereof.

The term “amsacrine” or m-amsa, as used herein means a compound with the structure:

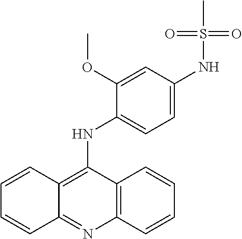

or a pharmaceutically acceptable solvate and/or prodrug thereof.

The term “analog” for example “daunorubicin analog” refers to a compound with a physical structure that is related to a parent compound e.g. ruboxyl (RBX) is a nitroxylated analog of daunorubicin.

The term “and/or” as used herein is meant to indicate that the listed options are either present together or individually. For example, the expression “pharmaceutically acceptable salt, solvate, and/or prodrug thereof” means that a compound can be a salt or a solvate or a prodrug of the referenced compound, or the compound can be a salt and a solvate and a prodrug of the referenced compound. For example, solvates of salts are alternate forms of compounds that are well known in the art.

For clarity, when a compound is referred to by its chemical name, unless otherwise indicated, this reference includes salts (where applicable), solvates and/or prodrugs of the compound. In an embodiment, when a compound is referred to by its chemical name, unless otherwise indicated, this reference includes salts (where applicable), and/or solvates of the compound.

The term “synergistic” as used herein means the enhanced or magnified effect of a combination on at least one property compared to the additive individual effects of each component of the combination. For example, compounds that induce cell death by the same mechanism, would not be expected to have more than additive effect. Synergism can be assessed and quantified for example by analyzing the Data by the Calcusyn median effect model where the combination index (CI) indicates synergism (CI<0.9), additively (CI=0.9-1.1) or antagonism (CI>1.1). Cis of <0.3, 0.3-0.7, 0.7-0.85, 0.85-0.90, 0.90-1.10 or >1.10 indicate strong synergism, synergism, moderate synergism, slight synergism, additive effect or antagonism, respectively. The CI is the statistical measure of synergy.

The term “cell death” as used herein includes all forms of cell death including necrosis and apoptosis.

As used herein, “contemporaneous administration” and “administered contemporaneously” means that the avermectin (e.g. IVM) and cytarabine and/or daunorubicin are administered to a subject such that they are each biologically active in the subject at the same time. The exact details of the administration will depend on the pharmacokinetics of the substances in the presence of each other, and can include administering one substance within 24 hours of administration of another, if the pharmacokinetics are suitable. Designs of suitable dosing regimens are routine for one skilled in the art. In particular embodiments, two substances will be administered substantially simultaneously, i.e. within minutes of each other, or in a single composition that comprises both substances.

The term “combination therapy” or “in combination with” as used herein means two or more substances, for example the avermectin (e.g. IVM) and a chemotherapeutic such as daunorubicin and/or cytarabine, are administered to a subject over a period of time, contemporaneously or sequentially e.g. the substances are administered at the same time or at different times within the period of time in a regimen that will provide beneficial effects of the drug combination, at similar or different intervals. For example, the combination therapy is intended to embrace co-administration, in a substantially simultaneous manner such as in a single capsule having a fixed ratio of active ingredients or in multiple, separate capsules for each substance. The compounds may or may not be biologically active in the subject at the same time. As an example, a first substance is administered weekly, and a second substance administered every other week for a number of weeks. The exact details of the administration will depend on the pharmacokinetics of the two substances. Designs of suitable dosing regimens are routine for one skilled in the art.

As used herein, the phrase “dosage form” refers to the physical form of a dose for example comprising compounds of the disclosure, and includes without limitation tablets, including enteric coated tablets, caplets, gelcaps, capsules, ingestible tablets, buccal tablets, troches, elixirs, suspensions, syrups, wafers, liposomal formulations and the like. The dosage form may be solid or liquid. Liposomal formulations, can for example be used to administer multiple compounds at fixed ratios. Liposmal formulations include for example liposomal daunorubicin or liposomal doxorubicin formulations.

As used herein, the phrase “effective amount” or “therapeutically effective amount” means an amount effective, at dosages and for periods of time necessary to achieve the desired result. For example in the context or treating a hematological malignancy, an effective amount is an amount that for example induces remission, reduces tumor burden, and/or prevents tumor spread or growth compared to the response obtained without administration of the compound. Effective amounts may vary according to factors such as the disease state, age, sex, weight of the animal. The amount of a given compound that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

As used herein, the phrase “standard amount” of a chemotherapeutic, for example “standard amount of cytarabine” means for example an amount or dose of cytarabine as approved by a health regulatory agency, such as the Health Canada, the US Federal Drug Agency (FDA), or recommended in a standard treatment protocol for the treatment of a hematological malignancy for example as specified in the product insert. In an embodiment, the effective amount of the chemotherapeutic administered is less than standard amount. In an embodiment, the effective amount of the chemotherapeutic administered is the standard amount. In an embodiment, the effective amount of cytarabine is less than the standard amount. In an embodiment, the effective amount of cytarabine is the standard amount.

Similarly, the phrase “standard amount of daunorubicin” means for example an amount or dose of daunorubicin as approved by a health regulatory agency, such as Health Canada, the US FDA, or recommended in a standard treatment protocol for the treatment of a hematological malignancy, for example as specified in the product insert. In an embodiment, the effective amount of daunorubicin is less than the standard amount. In an embodiment, the effective amount of daunorubicin is the standard amount.

The term “hematological malignancy” or “hematological cancer” as used herein refers to cancers that affect blood cells and/or bone marrow cells, and includes for example including hematological cancer cells, leukemias, lymphomas and myelomas.

The term “hematological cancer cell” as used herein refers a cancerous cell of the blood and bone marrow lineages, including primary cells. Hematological cancer cells include for example leukemia cells such as leukemia cells represented by CEM, TEX, THP1, HL-60, RSV411, K562, Jurkat, U937, OCI-M2, OCI-AML2 and NB4 leukemia cell lines and cells phenotypically similar thereto, lymphoma cells such as lymphoma cells represented MDAY-D2 and cell phenotypically similar thereto, and multiple myeloma cells such as multiple myeloma cells represented by OPM2, KMS11, LP1, UTMC2, KSM18, KSM12, H929, JJN3 and OCIMy5 myeloma cell lines and cells phenotypically similar thereto. Hematological cancer cells also include chronic myelogenous leukemia cells, including cells representing the blast crises phases such as K562 and cells phenotypically similar thereto; AML cells such as represented by HL-60, K562, OCI-M2, and NB4 and cells phenotypically similar thereto, ALL cells such as represented by RSV411 and Jurkat and cells phenotypically similar thereto, and lymphoma cells such as represented by MDAY-D2 and cells phenotypically similar thereto.

The term “leukemia” as used herein means any disease involving the progressive proliferation of abnormal leukocytes found in hemopoietic tissues, other organs and usually in the blood in increased numbers. For example, leukemia includes acute myeloid leukemia (AML), acute lymphocytic leukemia (ALL) and chronic myelogenous leukemia (CML).

The term “lymphoma” as used herein means any disease involving the progressive proliferation of abnormal lymphoid cells. For example, lymphoma includes mantle cell lymphoma, Non-Hodgkin's lymphoma, and Hodgkin's lymphoma. Non-Hodgkin's lymphoma would include indolent and aggressive Non-Hodgkin's lymphoma. Aggressive Non-Hodgkin's lymphoma would include intermediate and high grade lymphoma. Indolent Non-Hodgkin's lymphoma would include low grade lymphomas.

The term “myeloma” and/or “multiple myeloma” as used herein means any tumor or cancer composed of cells derived from the hemopoietic tissues of the bone marrow. Multiple myeloma is also knows as MM and/or plasma cell myeloma.

The term “pharmaceutically acceptable” means compatible with the treatment of animals, in particular, humans.

The term “pharmaceutically acceptable salt” means an acid addition salt which is suitable for or compatible with the treatment of patients.

The term “pharmaceutically acceptable acid addition salt” as used herein means any non-toxic organic or inorganic salt of any basic compound. Basic compounds that form an acid addition salt include, for example, compound comprising an amine group. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, acid addition salts are more soluble in water and various hydrophilic organic solvents, and generally demonstrate higher melting points in comparison to their free base forms. The selection of the appropriate salt will be known to one skilled in the art.

The term “pharmaceutically acceptable basic addition salt” as used herein means any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline, alkylammonias or ammonia. The selection of the appropriate salt will be known to a person skilled in the art.

The formation of a desired compound salt is achieved using standard techniques. For example, the neutral compound is treated with an acid or base in a suitable solvent and the formed salt is isolated by filtration, extraction or any other suitable method.

The term “prodrug” as used herein refers to a derivative of an active form of a known compound or composition which derivative, when administered to a subject, is gradually converted to the active form to produce a better therapeutic response and/or a reduced toxicity level. In general, prodrugs will be functional derivatives of the compounds disclosed herein which are readily convertible in vivo into the compound from which it is notionally derived. Prodrugs include, without limitation, acyl esters, carbonates, phosphates, and urethanes. These groups are exemplary, and not exhaustive, and one skilled in the art could prepare other known varieties of prodrugs. Prodrugs may be, for example, formed with available hydroxy, thiol, amino or carboxyl groups For example, the available OH and/or NH₂ in the compounds of the disclosure may be acylated using an activated acid in the presence of a base, and optionally, in inert solvent (e.g. an acid chloride in pyridine). Some common esters which have been utilized as prodrugs are phenyl esters, aliphatic (C₈-C₂₄) esters, acyloxymethyl esters, carbamates and amino acid esters. In certain instances, the prodrugs of the compounds of the disclosure are those in which the hydroxy and/or amino groups in the compounds is masked as groups which can be converted to hydroxy and/or amino groups in vivo. Conventional procedures for the selection and preparation of suitable prodrugs are described, for example, in “Design of Prodrugs” ed. H. Bundgaard, Elsevier, 1985.

Where the compounds according to the disclosure possess more than one or more asymmetric centre, they may exist as “stereoisomers”, such as enantiomers and diastereomers. It is to be understood that all such stereisomers and mixtures thereof in any proportion are encompassed within the scope of the present disclosure. It is to be understood that while the stereochemistry of the compounds of the disclosure may be as provided for in any given compound shown herein, such compounds may also contain certain amounts (e.g. less than 20%, less than 10%, less than 5%) of compounds having alternate stereochemistry.

The term “phenotypically similar” refers to a cell type that exhibits morphological, physiological and/or biochemical characteristics similar to another cell type. For example, a cell that is phenotypically similar to an AML cell can include a cell that comprises Auer rods. As another example, U937 cells which are derived from a patient with lymphoma, show morphological similarity to monocytoid AML cells. As a further example the leukemia cell line NB4 differentiates similar to promyelocytic cells with all trans retinoic acid (ATRA) and thereby represents a “phenotypically similar” model of PML cells.

The term “solvate” as used herein means a compound or its pharmaceutically acceptable salt, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions.

The term “ROS biomarker” as used herein, refers to a gene whose expression is increased in response to avermectin induced ROS generation.

The term “subject” as used herein includes all members of the animal kingdom including mammals, and suitably refers to humans.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject with early stage myeloma can be treated to prevent progression or alternatively a subject in remission can be treated with a compound or composition described herein to prevent recurrence. Treatment methods comprise administering to a subject a therapeutically effective amount of a compound described herein and optionally consists of a single administration, or alternatively comprises a series of applications. For example, the compounds described herein may be administered at least once a week. However, in another embodiment, the compounds may be administered to the subject from about one time per week to about once daily for a given treatment. In another embodiment, the compound is administered twice daily. The length of the treatment period depends on a variety of factors, such as the severity of the disease, the age of the patient, the concentration, the activity of the compounds described herein, and/or a combination thereof. It will also be appreciated that the effective dosage of the compound used for the treatment or prophylaxis may increase or decrease over the course of a particular treatment or prophylaxis regime. Changes in dosage may result and become apparent by standard diagnostic assays known in the art. In some instances, chronic administration may be required. For example, the compounds are administered to the subject in an amount and for a duration sufficient to treat the patient.

It is to be understood that the terms as defined herein are intended to apply in all embodiments described.

II. Methods

It is demonstrated herein that ivermectin (IVM) displayed preclinical activity against hematological malignancies in vitro and delayed tumor growth in vivo at concentrations that appear pharmacologically achievable. Mechanistically, IVM-induced induced chloride influx, membrane hyperpolarization and generated reactive oxygen species. Furthermore, IVM synergized with chemotherapeutic treatment to kill leukemia cells but not normal cells, specifically IVM was demonstrated to synergize with cytarabine and synergize with daunorubicin.

As IVM synergized with cytarabine and daunorubicin in cell and animal studies it is expected that the same anti-tumor cell effect can be obtained with lower concentrations when combined and/or the combination would provide for example increased anti-tumor efficacy without increased toxicity.

Further several chemotherapeutics are used for treating hematological malignancies. For example, cytarabine is used for treating acute myeloid leukemia and for some lymphomas. Also for example, mitoxantrone, amsacrine, daunorubicin, idarubicin are used interchangeably in the treatment of AML. Therapeutic results with these compounds are similar when similar dose intensity is used. Further the mechanism of action of the compounds is the same and toxicity is similar (N Engl J Med. 2009 Sep 24;361(13):1301-3 (19776412[PMID]); J Clin Oncol. 2009 Jan. 1; 27(1):61-9 (19047294[PMID]), N Engl J Med. 2009 Sep. 24; 361(13):1249-59 (19776406[PMID]); Hematology. 2001; 5(5):359-367(11399635[PMID]).

Accordingly, an aspect of the disclosure includes a method of treating a hematological malignancy comprising administering to a subject in need thereof, an effective amount of an avermectin, in combination with a chemotherapeutic. In another embodiment, the chemotherapeutic is a hematological malignancy chemotherapeutic. In a further embodiment, the chemotherapeutic is a DNA damaging chemotherapeutic drug.

In an embodiment, the chemotherapeutic is an anthracycline. In an embodiment, the anthracycline is one known in the art for treating a hematological malignancy, for example, to treat leukemia, optionally AML. In an embodiment, the chemotherapeutic or anthracycline is daunorubicin or a daunorubicin analog. In another embodiment, the anthracycline is idarubicin or doxorubicin.

In another embodiment, the chemotherapeutic is an anthracenedione. In an embodiment, the anthracenedione is one known in the art for treating a hematological malignancy.

In a further embodiment, the chemotherapeutic or the hematological malignancy chemotherapeutic is selected from cytarabine, daunorubicin, doxorubicin, idarubicin, mitoxantrone, and amsacrine and mixtures thereof. In an embodiment, the chemotherapeutic is selected from doxorubicin, mitoxantrone, m-amsa (amsacrine), and idarubicin. Doxorubicin, mitoxantrone, m-amsa (amsacrine), idarubicin are related to daunorubicin, e.g. daunorubicin family members.

In an embodiment, the method of treating a hematological malignancy comprises administering to a subject in need thereof, an effective amount of an avermectin, in combination with an effective amount of cytarabine and/or daunorubicin.

In an embodiment, the avermectin is selected from ivermectin, invermectin, avermectin, abamectin, doramectin, eprinomectin and selamectin and mixtures thereof. In another embodiment, the avermectin is ivermectin (IVM). In another embodiment, the avermectin is an avermectin that generates radical oxygen species in a leukemia cell.

In an embodiment, the avermectin is not a prodrug.

In an embodiment, the method comprises administering an effective amount of IVM in combination with a chemotherapeutic. In a further embodiment, the chemotherapeutic is a hematological malignancy chemotherapeutic. In a further embodiment, the chemotherapeutic is DNA damaging chemotherapeutic drug.

In an embodiment, the chemotherapeutic is not a prodrug.

In an embodiment, the method comprises administering an effective amount of IVM in combination with cytarabine.

In an embodiment, the method comprises administering an effective amount of IVM in combination with daunorubicin.

In an embodiment, the compounds are administered in amounts that together are sufficient to treat the hematological malignancy.

In an embodiment, the effective amount of the avermectin, for example IVM, is administered before administering the effective amount of the chemotherapeutic, for example cytarabine and/or daunorubicin. In another embodiment, the effective amount of the avermectin, for example IVM is administered after administering the effective amount of the chemotherapeutic, for example cytarabine. In another embodiment the effective amount of the avermectin and the effective amount of the chemotherapeutic, for example cytarabine and/or daunorubicin, are administered contemporaneously.

In an embodiment, the compounds are administered in a single dose or in multiple applications, at similar or different intervals, for example IVM is administered daily and cytarabine and/or daunorubicin is administered once or twice weekly for a particular number or weeks. In another embodiment, daunorubicin is administered daily, for example for 3 days. In another embodiment, cytarabine is administered once or twice daily, for example for 3 to 7 days.

In a further embodiment, the disclosure includes a use of an avermectin, in combination with an effective amount of a chemotherapeutic, optionally a hematological malignancy chemotherapeutic, for the treatment of a hematological malignancy. In an embodiment, the avermectin is an avermectin that generates radical oxygen species in a leukemia cell. In another embodiment, the chemotherapeutic is a DNA damaging chemotherapeutic drug.

In a further embodiment, the disclosure includes a use of an avermectin, in combination with an effective amount of cytarabine and/or daunorubicin for the treatment of a hematological malignancy.

Also disclosed in another embodiment, is use of an avermectin, in combination with a chemotherapeutic, optionally a hematological malignancy chemotherapeutic, for the manufacture of a medicament for the treatment of a hematological malignancy.

Another embodiment includes a use of an avermectin, in combination with cytarabine and/or daunorubicin for the manufacture of a medicament for the treatment of a hematological malignancy.

In another embodiment, the avermectin is an avermectin that generates radical oxygen species in a leukemia cell. In yet another embodiment, the chemotherapeutic is a DNA damaging chemotherapeutic drug.

In another aspect the disclosure includes, a method of inducing cell death in a hematological cancer cell comprising contacting the cell with an avermectin in combination with a chemotherapeutic, optionally a hematological malignancy chemotherapeutic. The contact is for example for a suitable length of time and for under suitable conditions to induce cell death.

In another embodiment, the method of inducing cell death in a hematological cancer cell comprises contacting the cell with an effective amount of the avermectin, e.g. IVM, and an effective amount of a chemotherapeutic for example, cytarabine and/or daunorubicin.

In an embodiment, the cell is in vitro. In another embodiment, the cell is in viva

In an embodiment, the chemotherapeutic is selected from cytarabine, daunorubicin, doxorubicin, idarubicin mitoxantrone, and amsacrine and mixtures thereof. In an embodiment chemotherapeutic is cytarabine and/or daunorubicin.

In an embodiment, IVM comprises at least 80% H₂B₁ₐ and less than 20% H₂B_{1b.} In another embodiment, IVM comprises at least 90% H₂B₁ₐ and less than 10% H₂B_{1b.}

In another embodiment, the hematological malignancy is leukemia. In another embodiment, the leukemia is selected from acute myeloid leukemia (AML), acute lymphocytic leukemia (ALL) and chronic myelogenous leukemia (CML). In an embodiment, the hematological cancer cell is leukemic cell, an AML cell, an ALL cell or a CML cell.

In a further embodiment, the hematological malignancy is a myeloma. In another embodiment, the hematological cancer cell is a myeloma cell.

In yet a further embodiment, the hematological malignancy is a lymphoma. In an embodiment, the hematological cancer cell is a lymphoma cell.

In an embodiment, compounds in the methods and uses described herein are comprised in a composition, dosage or dosage form described herein.

Changes in ROS production are indicative of a biological response to ivermectin. Genes upregulated as a result of ROS production may be used as biomarkers to monitor the biological response to for example an avermectin such as an ivermectin, as well as for determining the therapeutic range of avermectin, for example, ivermectin, in treating hematological malignancies when combined with an effective amount of a chemotherapeutic.

Accordingly, an aspect of the disclosure is a method of determining an avermectin activity in a subject or on a population of cells which comprises: administering to the subject or population of cells, an effective amount of an avermectin; determining a level of a ROS biomarker or a plurality of ROS biomarkers in a post-administration sample from the subject or population of cells and comparing the level of each biomarker in the post-administration sample with a base-line level, wherein an increase in the ROS biomarker level in the post-administration sample compared to the baseline level is indicative of avermectin activity sufficient to induce a biological response.

In an embodiment, the base line level is determined in a sample obtained from the subject prior to the administering step. In an embodiment, the avermectin is administered in combination with an effective amount of a chemotherapeutic. For example, the method can be used to determine and/or confirm sufficient dosing levels, for example in a clinical trial.

In an embodiment, the base line level is determined in a sample of the population of cells (e.g. comprising all or part of the population of cells). When the population of cells is a population of leukemia cells, such method can be used for example to determine if an avermectin is an avermectin that generates ROS in a leukemia cell.

In an embodiment, the ROS biomarker is selected from STAT1A, STAT1 B, TRIM22, OAS1 and IFIT3 and/or combinations thereof.

In a further embodiment, the ROS biomarker level in the post-administration sample compared to the baseline level is increased, for example, 2-fold or more, 3-fold or more, 4-fold or more, 5-fold or more, 7-fold or more, 10-fold or more, 20-fold or more, or 50-fold or more.

### III. Compositions

An aspect of the disclosure includes a composition comprising an avermectin, and a chemotherapeutic such as cytarabine and/or daunorubicin and optionally a suitable carrier or vehicle.

In an embodiment, the chemotherapeutic is a hematological malignancy chemotherapeutic. In yet another embodiment, the chemotherapeutic is DNA damaging chemotherapeutic drug.

In an embodiment, the chemotherapeutic is an anthracycline. In an embodiment, the anthracycline is one known in the art for treating a hematological malignancy. In another embodiment, the chemotherapeutic is an anthracenedione. In an embodiment, the anthracenedione is one known in the art for treating hematological malignancies. In another embodiment, the chemotherapeutic is a daunorubicin analog. In a further embodiment, the chemotherapeutic is selected from cytarabine, daunorubicin, doxorubicin, idarubicin mitoxantrone, and amsacrine and mixtures thereof.

In an embodiment, the composition comprises an effective amount of avermectin, and an effective amount of the chemotherapeutic such as cytarabine and/or daunorubicin and optionally a suitable carrier or vehicle.

In another aspect, the composition comprising an effective amount of an avermectin, and an effective amount of cytarabine and a suitable carrier or vehicle.

In another aspect, the composition comprises an effective amount of an avermectin, and an effective amount of daunorubicin and a suitable carrier or vehicle.

In another aspect, the composition comprises an effective amount of an avermectin, and an effective amount of a chemotherapeutic such as a hematological chemotherapeutic, for example cytarabine and/or daunorubicin and optionally a suitable carrier or vehicle for treating a hematological malignancy.

In another aspect, the composition comprises an effective amount of an avermectin, and an effective amount of cytarabine and a suitable carrier or vehicle for treating a hematological malignancy.

In another aspect, the composition comprising an effective amount of an avermectin, and an effective amount of daunorubicin and a suitable carrier or vehicle for treating a hematological malignancy.

In an embodiment, the avermectin is an avermectin that generates radical oxygen species in a leukemia cell.

In an embodiment, the avermectin is ivermectin, invermectin, avermectin, abamectin, doramectin, eprinomectin and selamectin and mixtures thereof. In another embodiment, the avermectin is ivermectin (IVM).

In an embodiment, the avermectin is IVM. In an embodiment, IVM comprises at least 80% H₂B₁ₐ and less than about 20% H₂B_{1b.} In another embodiment, IVM comprises at least 90% H₂B₁ₐ and less than 10% H₂B_{1b}

The compounds are suitably formulated into pharmaceutical compositions for administration to human subjects in a biologically compatible form suitable for administration in vivo.

The compositions described herein can be prepared by per se known methods for the preparation of pharmaceutically acceptable compositions that can be administered to subjects, such that an effective quantity of the active substance is combined in a mixture with a pharmaceutically acceptable vehicle.

Suitable vehicles are described, for example, in Remington's Pharmaceutical Sciences (2003-20^{th} Edition). On this basis, the compositions include, albeit not exclusively, solutions of the substances in association with one or more pharmaceutically acceptable vehicles or diluents, and contained in buffered solutions with a suitable pH and iso-osmotic with the physiological fluids.

Pharmaceutical compositions include, without limitation, lyophilized powders or aqueous or non-aqueous sterile injectable solutions or suspensions, which optionally further contain antioxidants, buffers, bacteriostats and solutes that render the compositions substantially compatible with the tissues or the blood of an intended recipient. Other components that are optionally present in such compositions include, for example, water, surfactants (such as Tween™), alcohols, polyols, glycerin and vegetable oils. Extemporaneous injection solutions and suspensions may be prepared from sterile powders, granules, tablets, or concentrated solutions or suspensions. The composition can be supplied, for example but not by way of limitation, as a lyophilized powder which is reconstituted with sterile water or saline prior to administration to the subject.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesyl-phosphotidyl-ethanolamine (DOPE), and liposomes. Such compositions should contain a therapeutically effective amount of the compound(s), together with a suitable amount of carrier so as to provide the form for direct administration to the subject.

In an embodiment, the compositions described herein are administered for example, by parenteral, intravenous, subcutaneous, intramuscular, intracranial, intraorbital, ophthalmic, intraventricular, intracapsular, intraspinal, intracisternal, intraperitoneal, intranasal, aerosol or oral administration.

Compositions for nasal administration can conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochiorohydrocarbon. The aerosol dosage forms can also take the form of a pump-atomizer.

Wherein the route of administration is oral, the dosage form may be for example, incorporated with excipient and used in the form of enteric coated tablets, caplets, gelcaps, capsules, ingestible tablets, buccal tablets, troches, elixirs, suspensions, syrups, wafers, and the like. The oral dosage form may be solid or liquid.

Accordingly, a further aspect of the disclosure is a composition formulated for as an oral dosage form selected from enteric coated tablets, caplets, gelcaps, and capsules, each unit dosage form about 1 to less than about 500 mg, suitably about 1 to about 350 mg, about 1 to about 150 mg, about 1 to about 120 mg, about 1 to about 100 mg, about 1 to about 80 mg, about 1 to about 50 mg, about 1 to about 30 mg, about 5 to about 350 mg, about 5 to about 150 mg, about 5 to about 120 mg, about 5 to about 100 mg, about 5 to about 80 mg, about 5 to about 50 mg, about 5 to about 30 mg, about 3 to about 30 mg, or about 3.5 to about 5 mg, of an avermectin, and an effective amount of cytarabine and/or daunorubicin and optionally a suitable carrier or vehicle.

In an embodiment, the disclosure includes a pharmaceutical composition wherein the dosage form is a solid dosage form. A solid dosage form refers to individually coated tablets, capsules, granules or other non-liquid dosage forms suitable for oral administration. It is to be understood that the solid dosage form includes, but is not limited to, modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. It is also possible to freeze-dry the compounds described herein and use the lyophilizates obtained, for example, for the preparation of products for injection.

Accordingly, a further aspect of the disclosure is a pharmaceutical composition in solid dosage form about 1 to less than about 500 mg, suitably about 1 to about 350 mg, about 1 to about 150 mg, about 1 to about 120 mg, about 1 to about 100 mg, about 1 to about 80 mg, about 1 to about 50 mg, about 1 to about 30 mg, about 5 to about 350 mg, about 5 to about 150 mg, about 5 to about 120 mg, about 5 to about 100 mg, about 5 to about 80 mg, about 5 to about 50 mg, about 5 to about 30 mg, about 3 to about 30 mg, or about 3.5 to about 5 mg, of an avermectin, and an effective amount of cytarabine and/or daunorubicin and optionally a suitable carrier or vehicle.

Compositions suitable for buccal or sublingual administration include tablets, lozenges, and pastilles, wherein the active ingredient is formulated with a carrier such as sugar, acacia, tragacanth, and/or gelatin and/or glycerin.

In another embodiment, the disclosure describes a pharmaceutical composition wherein the dosage form is a liquid oral dosage form. A person skilled in the art would know how to prepare suitable formulations. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999.

Accordingly, a further aspect of the disclosure is a pharmaceutical composition in oral liquid dosage form comprising about 1 to less than about 500 mg, suitably about 1 to about 350 mg, about 1 to about 150 mg, about 1 to about 120 mg, about 1 to about 100 mg, about 1 to about 80 mg, about 1 to about 50 mg, about 1 to about 30 mg, about 5 to about 350 mg, about 5 to about 150 mg, about 5 to about 120 mg, about 5 to about 100 mg, about 5 to about 80 mg, about 5 to about 50 mg, about 5 to about 30 mg, about 3 to about 30 mg, or about 3.5 to about 5 mg, of an avermectin and an effective amount cytarabine and/or daunorubicin and optionally a suitable carrier or vehicle.

In another embodiment, the disclosure describes a pharmaceutical composition wherein the dosage form is an injectable dosage form. An injectable dosage form is to be understood to refer to liquid dosage forms suitable for, but not limited to, intravenous, subcutaneous, intramuscular, or intraperitoneal administration. Solutions of compounds described herein can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999.

Accordingly, a further aspect of the disclosure is a pharmaceutical composition in injectable dosage form comprising about 1 to less than about 500 mg, suitably about 1 to about 350 mg, about 1 to about 150 mg, about 1 to about 120 mg, about 1 to about 100 mg, about 1 to about 80 mg, about 1 to about 50 mg, about 1 to about 30 mg, about 5 to about 350 mg, about 5 to about 150 mg, about 5 to about 120 mg, about 5 to about 100 mg, about 5 to about 80 mg, about 5 to about 50 mg, about 5 to about 30 mg, about 3 to about 30 mg, or about 3.5 to about 5 mg of an avermectin, and an effective amount of cytarabine and/or daunorubicin and optionally a suitable carrier or vehicle.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists.

In another embodiment, the dosage form can alternatively comprise about 0.01 to about 20 mg of an avermectin/kg body weight, about 0.02 to about 10 mg of an avermectin/kg body weight, about 0.2 to about 10 mg of an avermectin/kg body weight, about 0.05 to about 5 mg of an avermectin/kg body weight about 0.05 to about 2.5 mg of an avermectin/kg body weight, or about 0.05 to about 1.5 mg of an avermectin/kg body weight of a subject in need of such treatment formulated into a solid oral dosage form, a liquid oral dosage form, or an injectable dosage form. In an embodiment, the dosage comprises about 0.05 to about 1.5 mg of an avermectin/kg body weight of a subject in need of such treatment formulated into a solid oral dosage form, a liquid dosage oral form, or an injectable dosage form.

In an embodiment, the composition comprises a pharmaceutical combination.

In an embodiment, the pharmaceutical composition comprises separate formulations of the avermectin and the chemotherapeutic. In an embodiment, the composition comprises a single formulation.

In an embodiment the dosage, is a daily dosage.

The avermectin, and the effective amount of cytarabine and/or daunorubicin are optionally in the same dosage form or in different dosage forms. For example the avermectin (e.g. IVM) is in an oral dosage form and the cytarabine and/or daunorubicin is an injectable dosage form.

### III. Kits and Packs

Another aspect of the disclosure includes a kit. In an embodiment, the kit comprises an avermectin and instructions for administering in combination with and cytarabine and/or daunorubicin. In another embodiment, the kit comprises an avermectin and/or cytarabine and/or daunorubicin and instructions for administering in combination with the other(s). In an embodiment, the kit is for use in treating a hematological malignancy in a subject. The kit may comprise any of the therapeutic combinations of the disclosure. The kits may be tailored to the needs of types of patients or other clinically relevant factors such as age, body weight, responsiveness or non-responsiveness to prior treatments, etc.

Another aspect of the disclosure includes a pack e.g. a pharmaceutical pack. The compositions and/or formulations described herein for treating a hematological malignancy can be in comprised in a pharmaceutical pack. In an embodiment the pharmaceutical pack comprises an avermectin and cytarabine and/or daunorubicin and instructions for administering for example a daily dose.

In an embodiment, the kit or pack comprises separate formulations of the avermectin and the chemotherapeutic. In an embodiment, the kit or pack comprises a single formulation of the avermectin and the chemotherapeutic. In an embodiment, the kit or pack comprises IVM.

The following non-limiting examples are illustrative of the present disclosure:

### EXAMPLES

### Example 1

### Materials and Methods

### Reagents

The compounds in the chemical library were purchased from Sigma Aldrich (St. Louis, Mo.). Annexin V-FITC, Propidium Iodide (PI) were purchased from Biovision (Mountain View, Calif.). Indo-1 AM, 6-methoxy-N-(3-sulfopropryl)quinolinium (SPQ), carboxydichiorofluorescein diacetate (Carboxy H₂DCF-DA), bis-(1,3-dibutylbarbituric acid)trimethine oxonol (DiBAC₄(3) and 5,5′,6,6′-tetrachloro-1,1′,3,3′-tetraethyl benzimidazolylcarbocyanine iodide (JC-1) were all purchased from Invitrogen Canada (Burlington, Canada).

### Cell Lines

Human leukemia (OCI-AML2, HL60, K562, KG1a, U937) and murine (MDAY-D2) leukemia cell lines and prostate cancer (DU145 and PPC-1) cell lines were maintained in RPMI 1640 medium. Media was supplemented with 10% fetal calf serum (FCS), 100 μg/mL penicillin and 100 units/mL of streptomycin (all from Hyclone, Logan, Utah). TEX human leukemia cells were maintained in IMDM, 15% FBS, 2 mM L-glutamine, 1%, penicillin-streptomycin, 20 ng/mL SCF, 2 ng/mL IL-3. All cells were incubated at 37° C. in a humidified air atmosphere supplemented with 5% CO₂.

Primary Cells

Primary human acute myeloid leukemia (AML) samples were isolated from fresh bone marrow and peripheral blood samples of consenting patients. Similarly, primary normal hematopoietic cells were obtained from healthy consenting volunteers donating peripheral blood mononuclear cells

(PBSCs) for stem cell transplantation. The mononuclear cells were isolated from the samples by Ficoll density centrifugation. Primary cells were cultured at 37° C. in IMDM supplemented with 20% FCS and appropriate antibiotics.

### Chemical Screen for Cytotoxic Compounds

HL60, KG1a, and OCI-AML2 leukemia cells were seeded into 96-well polystyrene tissue culture plates (Nunc). After seeding, cells were treated with aliquots of the chemical library (=100) at increasing concentrations (3 to 50 μM) with a final DMSO concentration of 0.5%. Seventy two hours after incubation, cell growth and viability was measured by the MTS assay. Liquid handling was performed by a Biomek FX Laboratory Automated Workstation (Beckman Coulter Fullerton, Calif.).

### Cell Viability Assays

Cell growth and viability was assessed by the MTS assay (Promega, Madison, Wis.) according to the manufacturer's instructions and as previously described.¹² Cell death was measured by Annexin V-fluoroscein isothiocyanate (FITC) and Propidium Iodide (PI; Biovision Research Products, Mountain View, Calif.) staining and flow cytometry according to the manufacturer's instructions and as previously described.¹³ To identify CD34+ cells, a normal PBSC sample was co-stained with PE-anti-CD34+ (Beckman Coulter, Marseille France), and APC-anti-CD45 (Becton Dickenson, San Jose Calif.).

To assess clonogenic growth, primary AML cells or granulocyte colony-stimulating factor (G-CSF) mobilized PBSCs (4×10⁵/mL) were treated with IVM or buffer control for 24 hours. After treatment, cells were washed and 10⁵ cell/mL were plated in duplicate in MethoCult GF H4434 medium (StemCell Technologies, Vancouver, BC) containing 1% methycellulose in IMDM, 30% FCS, 1% bovine serum albumin, 3 U/mL of recombinant human erythropoietin, 10⁻⁴ M of 2-mercaptoethanol, 2 mM of L-glutamine, 50 ng/mL of recombinant human stem cell factor, 10 ng/mL of GM-CSF, and 10 ng/mL of rh IL-3. Alternatively, primary cells were plated directly into MethoCult GF H4434 medium with ivermectin. Seven days (AML samples) or 14 days (normal PBCS) after plating, the number of colonies containing 10 or more cells for AML or over 100 cells for normal samples was counted as previously described.¹⁴

### Cell Cycle Analysis

Cell cycle analysis was performed as described previously.¹⁵ Briefly, cells were harvested, washed with cold PBS, and resuspended in 80% cold ethanol. Cells were then treated with 100 ng/mL DNase-free RNase A (Invitrogen) at 37° C. for 30 min, washed with cold PBS, and resuspended in PBS with 50 μg/mL propidium iodide. DNA content was analyzed by flow cytometry (FACSCalibur; Becton Dickinson). The percentage of cells in each phase of the cell cycle was calculated with FlowJo version 8.8 (TreeStar, Ashland, Oreg.).

### Assessment of Ivermectin's Anticancer Activity in Mouse Models of Leukemia

MDAY-D2 murine leukemia cells, and K562 and OCI-AML2 human leukemia cells (2.5×10⁵) were injected subcutaneously into both flanks of sub-lethally irradiated (3.5 Gy) NOD/SCID mice (Ontario Cancer

Institute, Toronto, ON). Four (OCI-AML2), five (MDAY-D2), or seven (K562) days after injection, once tumors were palpable, mice were then treated daily for 10 days (K562) or treated with 8 doses over 10 days (OCI-AML2) with IVM (3 mg/kg) by oral gavage in water or vehicle control (n=10 per group). MDAY-D2 mice were treated similarly but dosage escalated from 3 mg/kg (4 days) to 5 mg/kg (3 days) and 6 mg/kg (3 days) as the drug was well tolerated. Tumor volume (tumor length x width^{2×0.5236}) was measured three times a week using calipers. Fourteen (MDAY-D2), 15 (OCI-AML2) or 17 (K562) days after injection of cells, mice were sacrificed, tumors excised and the volume and weight of the tumors were measured.

In order to measure gene expression changes in vivo, OCI-AML2 cells (2.5×10⁵) were injected subcutaneously into the flanks of sub-lethally irradiated NOD/SCID mice. Once tumors were established, mice were treated with ivermectin (7 mg/kg) or vehicle control intraperitoneally for 5 days. After treatment, mice were sacrificed, and tumors harvested. mRNA was extracted and changes in STAT expression were measured by quantitative RT-PCR (QRT-PCR). Evidence of apoptosis was measured by Tunel staining and immunohistochemistry (Pathology Research Program, University Health Network, Toronto, Canada).

### Intracellular Ion Measurements

Intracellular chloride concentration was measured using a fluorescent indicator for chloride, SPQ as previously described.¹⁶ Upon binding halide ions like chloride, SPQ is quenched resulting in a decrease in fluorescence without a shift in wavelength. After treating OCI-AML2 (5×10⁵) cells and DU145 (4×10⁵) overnight with IVM (3 to 10 μM), cells were incubated for 15 minutes with SPQ (5 mM) at 37° C. in a hypotonic solution (HBSS/H2O 1:1) to promote the intracellular uptake of SPQ. After 15 minutes of incubation with SPQ, cells were diluted 15:1 in HBSS and centrifuged. The supernatant was removed, cells were resuspended in 200 μl of fresh HBSS and incubated for 15 minutes in 37° C. to allow recovery from the hypotonic shock. Cells were then stained with propidium iodide and SPQ fluorescence in the PI negative cells was determined using an LSR-II flow cytometer (Beckton Dickonson, San Jose, Calif.) (excitation 351 nm, emission 485 nm). In parallel, changes in cell size were determined by measuring forward light scatter by flow cytometry. Results were analyzed with FlowJo version 8.8 (TreeStar, Ashland, Oreg.).

Changes in cytosolic calcium concentration were detected with the fluorescent dye, Indo-1 AM (final concentration 6 μM) as previously described. ¹⁷

### Determination of Plasma Membrane Potential

Plasma membrane potential was measured as previously described.¹⁸ Briefly, cells treated with IVM or buffer control in RPM1, chloride replete medium (140 mM sodium chloride, 5 mM potassium chloride, 1 mM magnesium sulfate, 1.8 mM calcium acetate, 10 mM glucose, 10 mM HEPES and 0.1% (wt/v) BSA), or chloride free media where equimolar gluconate salts of sodium and potassium replaced the sodium chloride in the chloride replete medium. After incubation, cells were stained with DiBAC₄(3) (final concentration 30 nM) and fluorescence determined by flow cytometry (BD FACS Canto, Becton Dickinson, San Jose, Calif.) (excitation=488 nm, emission=516 nm) Analysis was conducted using FACSDiva Software (BD Biosciences). Calibration curves were prepared using phosphate buffers with varying potassium ion concentrations as previously described.¹⁸

To measure mitochondrial membrane potential, cells were treated with IVM similarly as described above and then washed twice with PBS and incubated with 2 μM of 5,5′,6,6′-tetrachloro-1,1′,3,3′-tetraethyl benzimidazolylcarbocyanine iodide (JC-1, Invitrogen) for 20 minutes at 37° C. Each sample was then washed twice with 1 mL PBS and resuspended in 300 μL PBS prior to being read on a BD FACS Canto. Samples were excited at 488 nm and emission was collected at 526 nm (green) and 595 nm (red). Analysis was conducted using FACSDiva Software (BD Biosciences). To obtain the mitochondrial membrane potential (red/green), emission from the red channel was divided by emission from the green channel.

### Detection of Reactive Oxygen Species

Intracellular Reactive oxygen species (ROS) were detected by staining cells with Carboxy-H₂DCFDA (final concentration 10 μM) and flow cytometric analysis as previously described.²⁰ Cells were treated with IVM, cytarabine and daunorubicin overnight and stained with Carboxy-H₂DCFDA in PBS buffer at 37° C. for 30 minutes and simultaneously, cells were stained with propidium iodide to identify viable cells and assess their reactive oxygen intermediate levels. Data were analyzed with FlowJo version 8.8 (TreeStar, Ashland, Oreg.).

### Drug Combination Studies

The combination index (CI) was used to evaluate the interaction between IVM and cytarabine or daunorubicin. OCI-AML2 and U937 cells were treated with increasing concentrations of IVM, cytarabine and daunorubicin. Seventy-two hours after incubation cell viability was measured by the MTS assay. The Calcusyn median effect model was used to calculate the CI values and evaluate whether the combination of IVM with cytarabine or daunorubicin was synergistic, antagonistic or additive. CI values of <1 indicate synergism, CI=1 indicate additivity and CI>1 indicate antagonism.²¹

### Gene Expression Studies

Leukemia cells were treated with buffer control or ivermectin (3 μM) for 30 and 40 hours. After treatment, cells were harvested, total RNA was isolated. Total RNA (10 μg) was used for cRNA amplification using the Invitrogen SuperScript kit (Life Technologies, Inc., Burlington, ON, Canada). Amplification and biotin labeling of antisense cRNA was performed using the Enzo® BioArray™ High Yield™ RNA transcript labeling kit (Enzo Diagnostics, Farmingdale, N.Y., USA), according to the manufacturer's instructions. RNA was then hybridized to Affymetrix HG U133 Plus 2.0 gene expression oligonucleotide arrays (Affymetrix, Santa Clara, Calif., USA). Microarray slides were scanned using the GeneArray 2500 scanner (Agilent Technologies). Microarray data were analyzed using GeneSpring GX v10.0 (Agilent), and lists of genes deregulated>2-fold after 30 and 40 hr ivermectin treatment were derived. Pathways and gene ontology analyses were carried out using Ingenuity Pathways Analysis (www.ingenuity.com); and the Database for Annotation, Visualization and Integrated Discovery (DAVID; http://david.abcc.ncifcrf.gov). Data have been deposited into Array Express (E-MEXP-2528).

Gene expression changes were evaluated in OCI AML2, 0937, HL60, DU145 and PPC-1 cells by real-time Q-RT-PCRas described previously^{15, 47}. The expression level of STAT1A, STAT1B, TRIM22, OAS1 and IFIT3 before and after incubation of leukemia cells with ivermectin was evaluated.

### Results

A Chemical Screen Identifies Ivermectin with Potential Anti-Cancer Activity

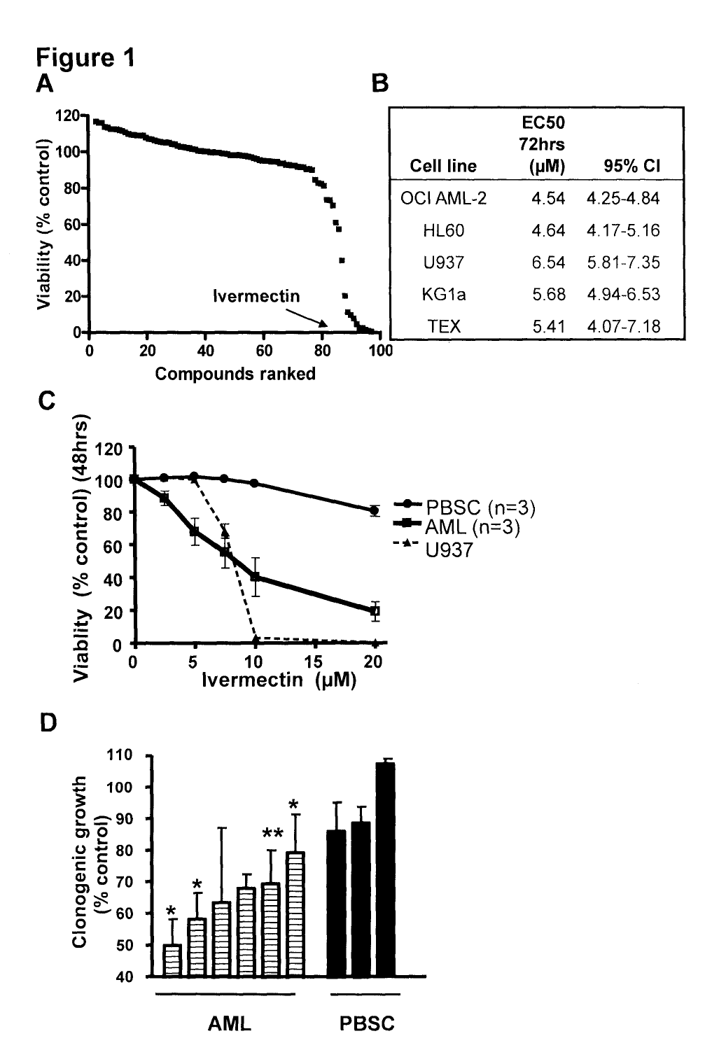

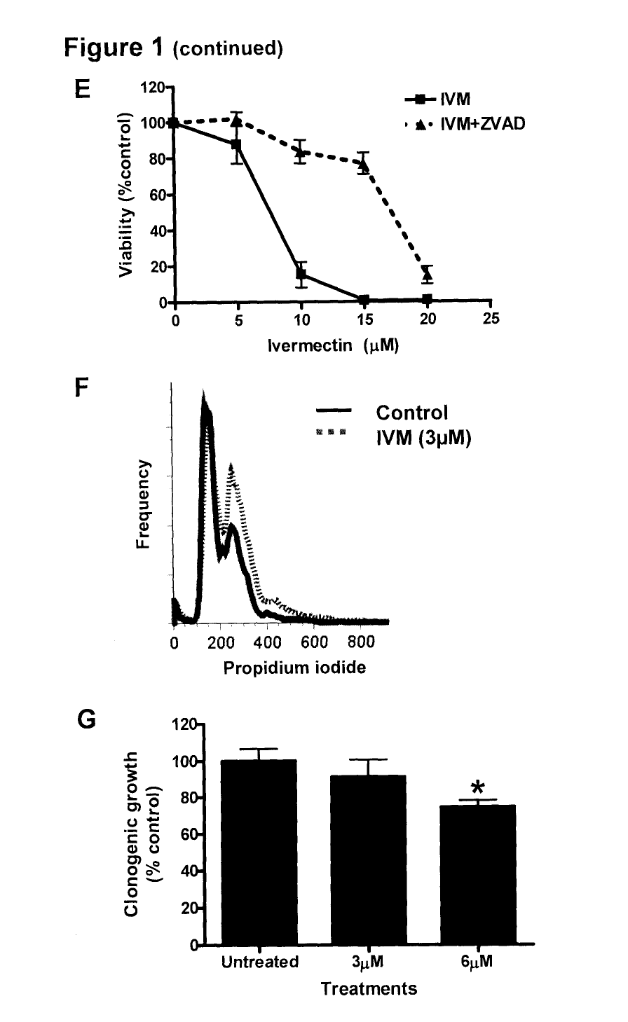

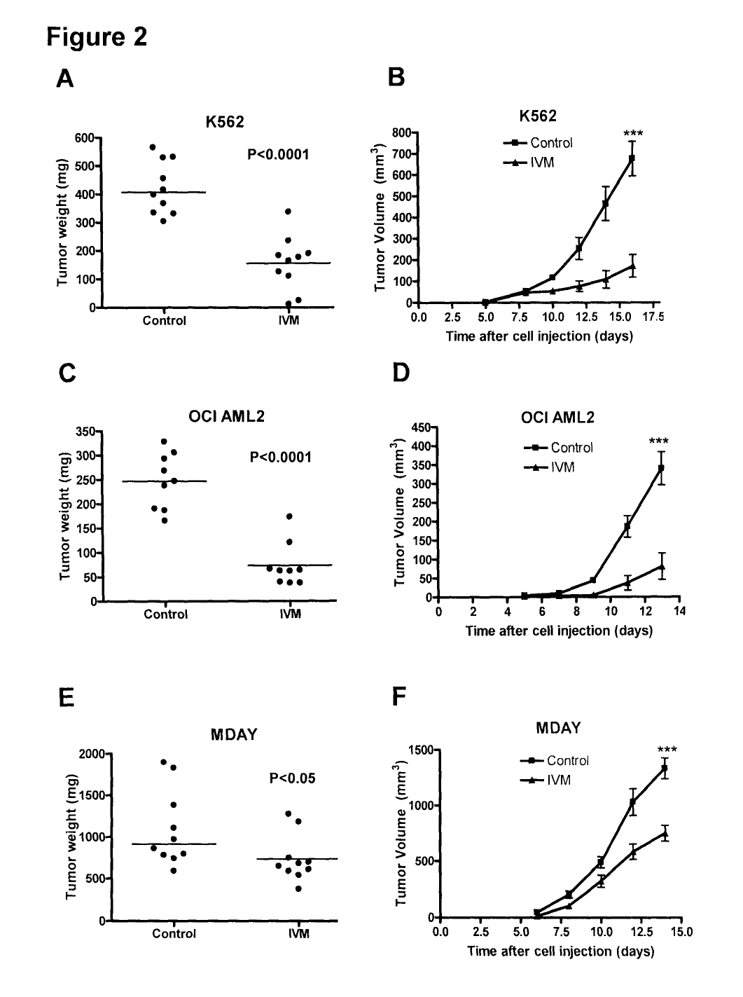

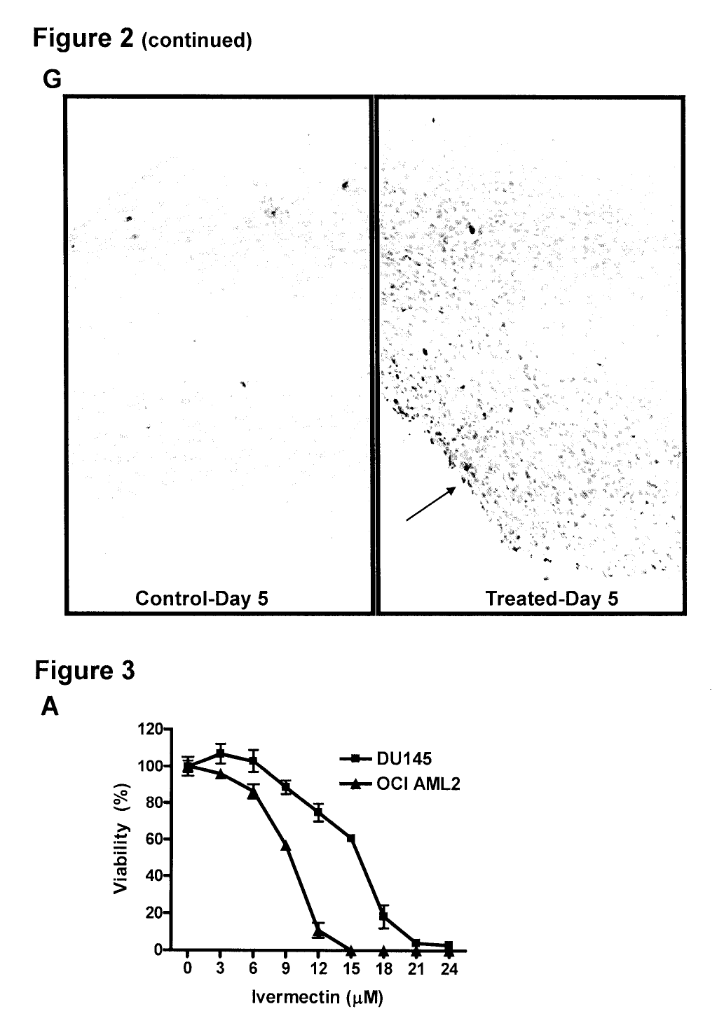

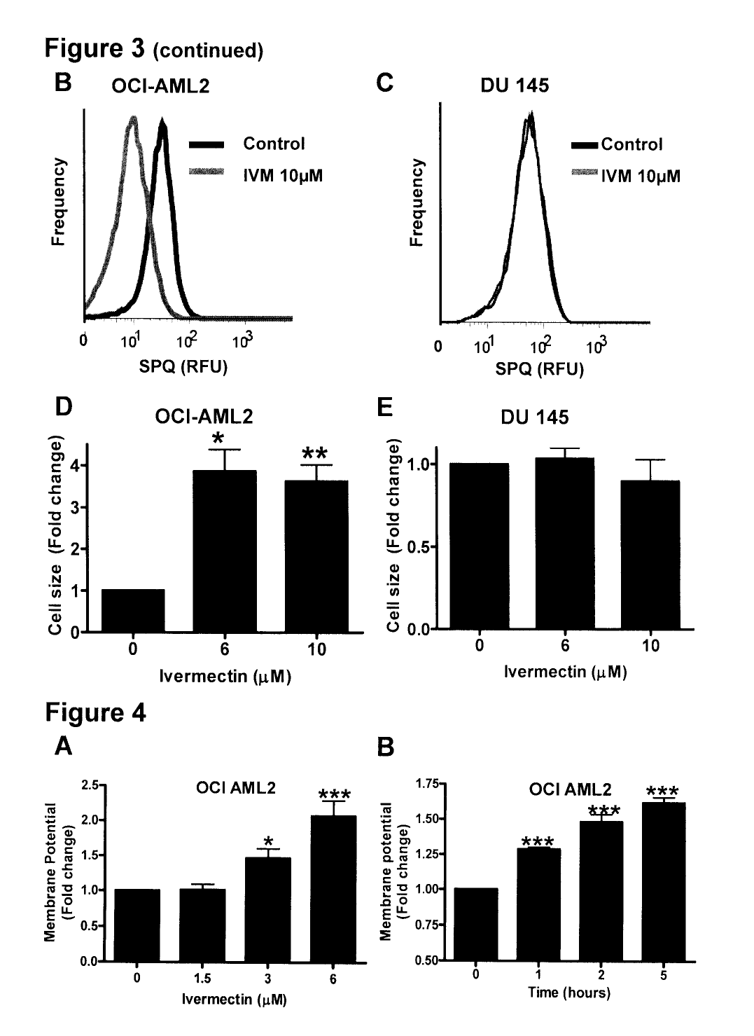

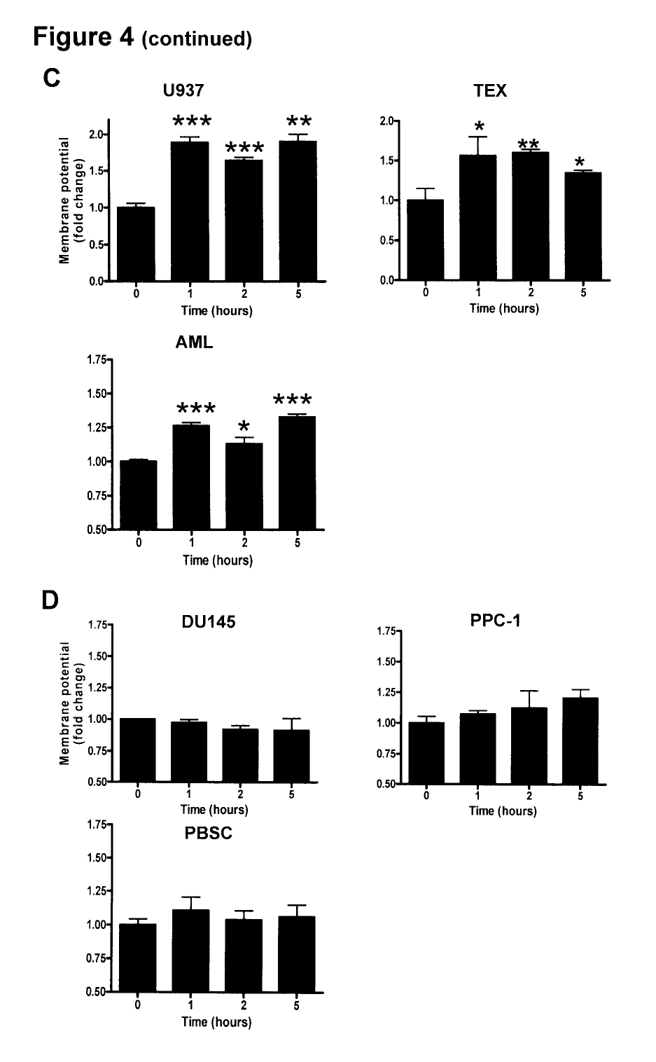

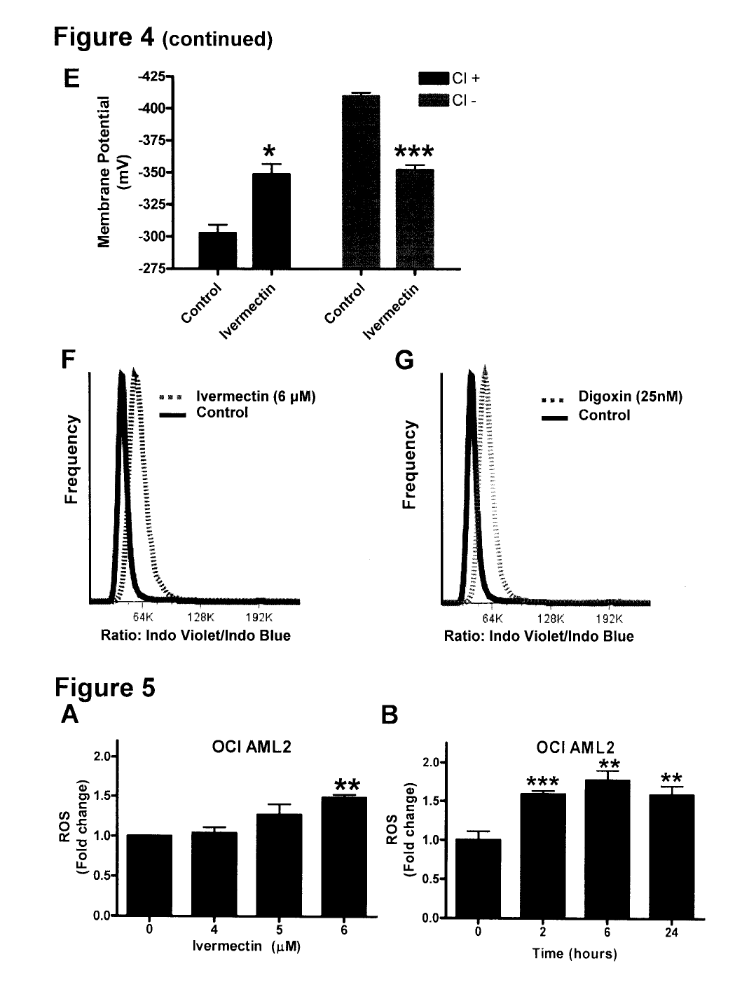

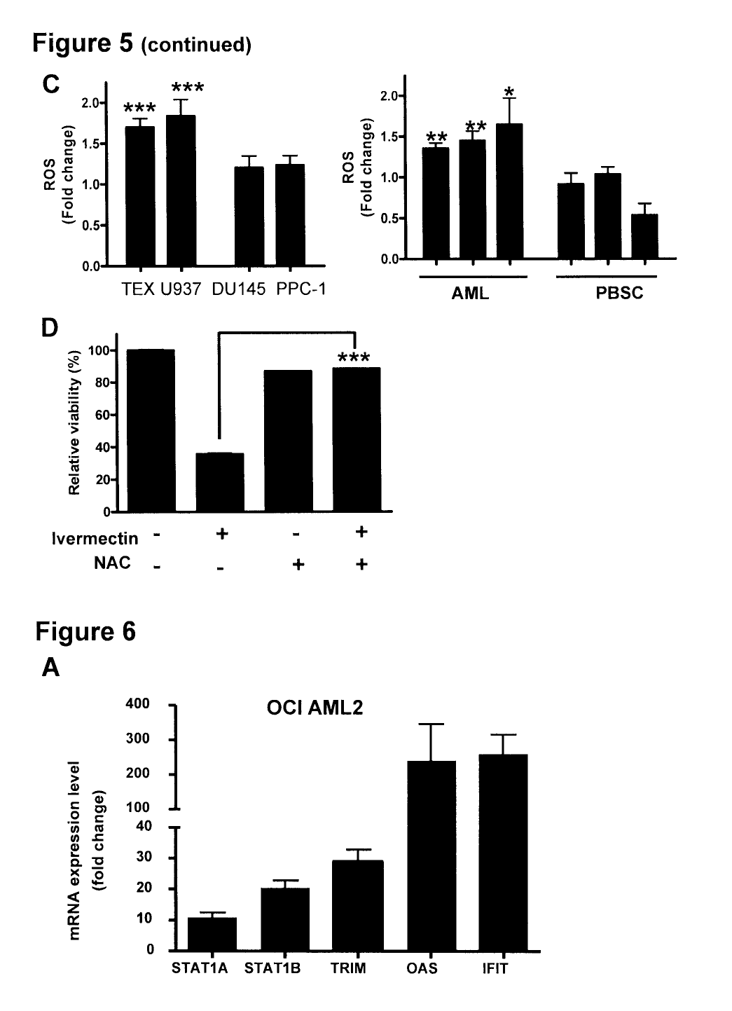

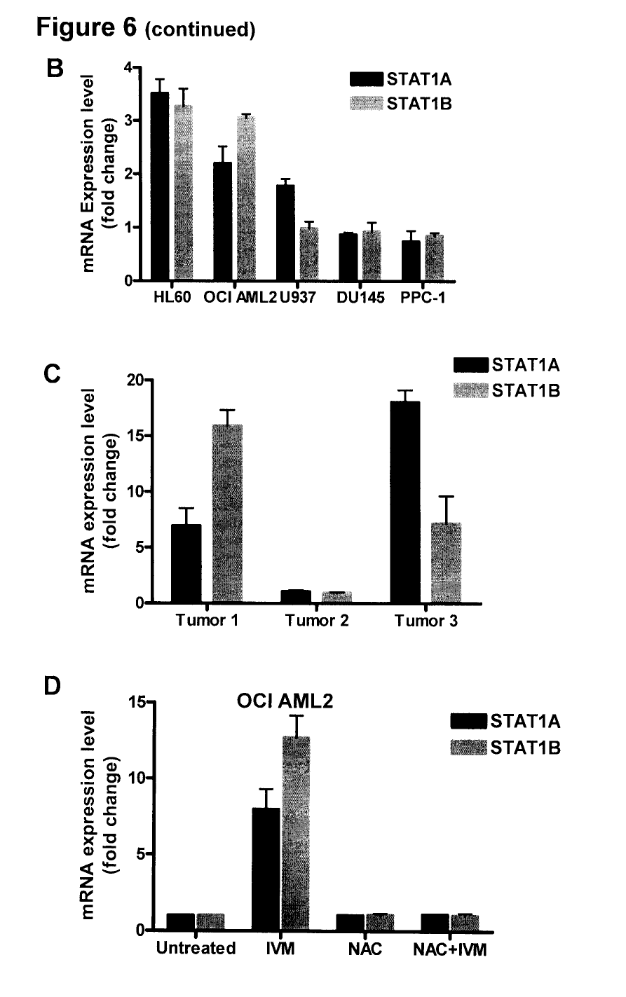

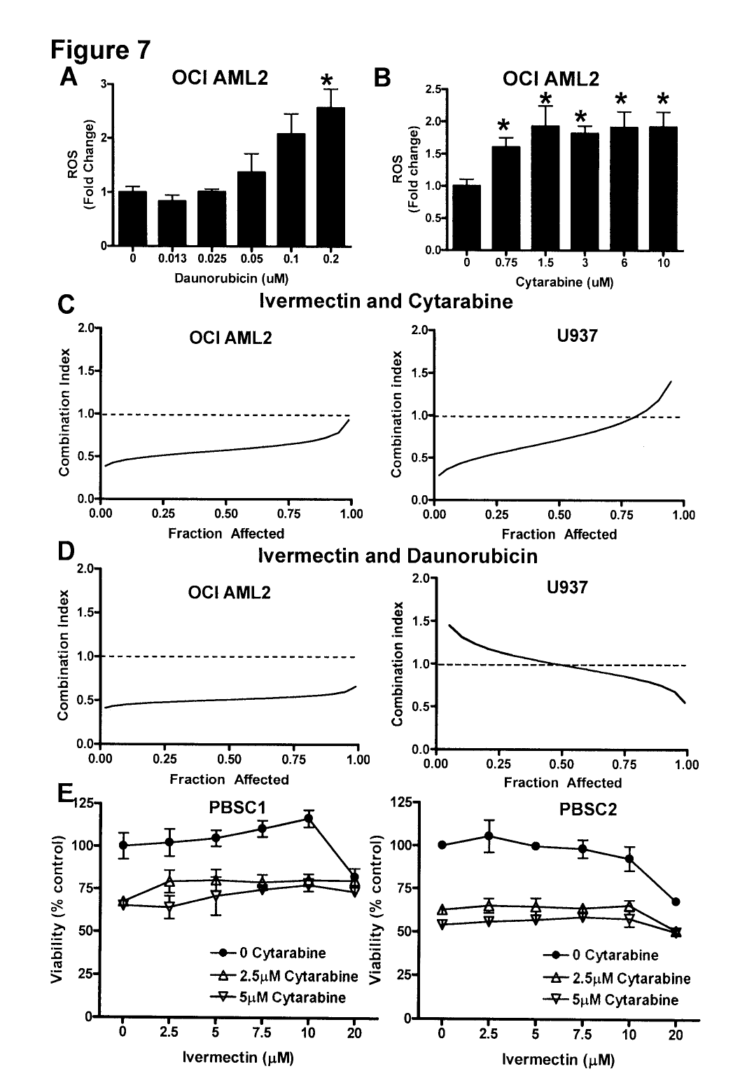

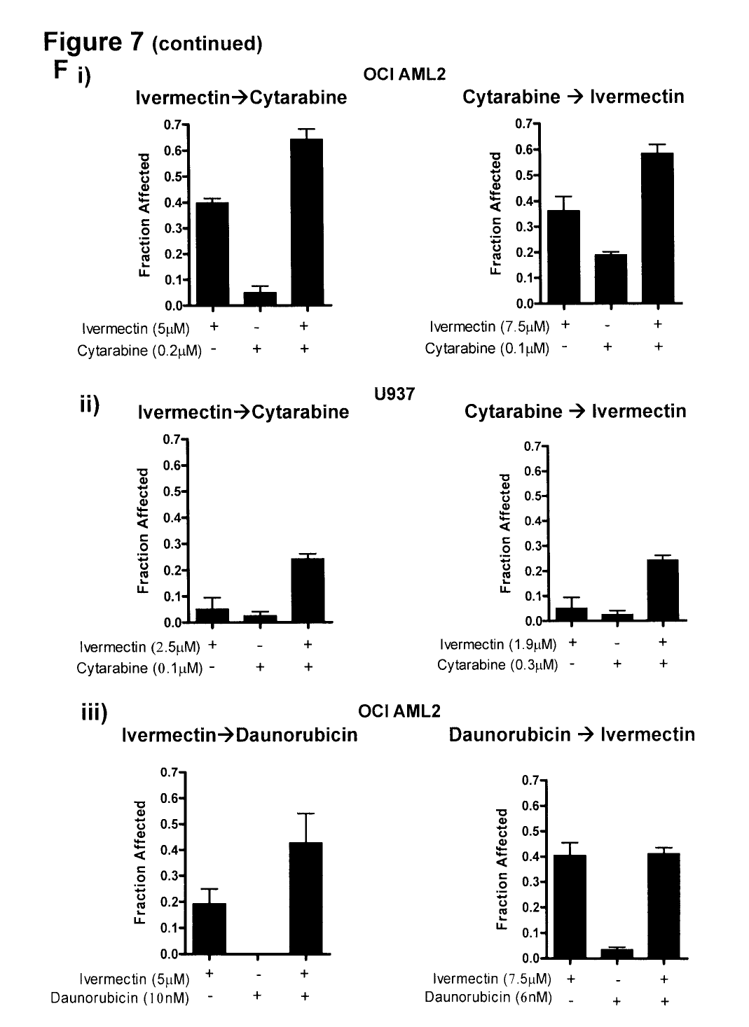

A small chemical library (n=100) focused on anti-microbials and metabolic regulators with wide therapeutic windows and well understood pharmacokinetics was compiled. OCI-AML2, HL60, and KG1a leukemia cell lines were treated with aliquots of this chemical library at five concentrations (3 to 50 μM). Seventy two hours after incubation, cell growth and viability was measured by the MTS assay. From this screen, IVM reduced cell viability in all cell lines in the screen with an EC50<10 μM. The results for the screen of OCI-AML2 cells with compounds added at a final concentration of 6 μM is shown in FIG. 1A.

### Ivermectin is Cytotoxic to Malignant Cell Lines and Primary Patient Samples

Having identified IVM in the chemical screens, the effects of IVM on cell growth and viability was tested in a panel of leukemia, cell lines. The effects of ivermectin on cell growth and viability in a panel of 5 leukemia cell lines were tested. Cells were treated with increasing concentrations of ivermectin and 72 hours after incubation, cell growth and viability were assessed by the MTS assay. Ivermectin decreased the viability of the tested leukemia cell lines with an EC50 of approximately 5 μM (FIG. 1B). The loss of viability was detected at 24 hours after treatment and increased in a time dependent manner. Cell death and apoptosis were confirmed by Annexin V and PI staining (FIG. 1C). Cell death was caspase-dependent, as co-treatment with the pan-caspase inhibitor z-VAD-fmk abrogated cell death (FIG. 1E). Furthermore, times and concentrations of ivermectin that preceded cell death induced G2 cell cycle arrest. (FIG. 1F).

Given the cytotoxicity of IVM toward leukemia cell lines, its cytotoxicity was compared to primary normal hematopoietic cells and acute myeloid leukemia (AML) patient samples (n=4 intermediate risk cytogenetics, n=1 good risk cytogenetics, and n=1 unknown cytogenetics). Normal hematopoietic cells and patient sample cells were treated for 48 hours with increasing concentrations of IVM. After incubation, cell viability was measured by Annexin V and PI staining. Ivermectin was cytotoxic to AML patient samples at low micromolar concentrations. In contrast, it did not induce cell death in the peripheral blood stem cells (PBSC) at concentrations up to 20 μM (FIG. 1C). However, when gating on the CD34+ cells from one PBSC sample, ivermectin induced cell death with an EC₅₀ of 10.5±0.6 mM. Thus, ivermectin induced cell death in primary AML cells preferentially over normal cells.

Ivermectin was also evaluated in clonogenic assays in primary normal hematopoietic and AML cells. Ivermectin (6 μM) had minimal effects on the clonogenic growth of normal hematopoetic cells (n=3) with <15% reduction in clonogenic growth. In contrast, ivermectin reduced clonogenic growth by ≧40% in 3/6 primary AML samples (FIG. 1D). Similar effects were noted when primary cells were directly plated into clonogenic assays with ivermectin (FIG. 1G).

### Ivermectin Delays Tumor Growth in Mouse Models of Leukemia

Given the effects of IVM as a potential anti-leukemic agent, IVM was evaluated in mouse models of leukemia. Human leukemia (OCI-AML2 and K562) and murine leukemia (MDAY-D2) cells (MDAY-D2, OCI-AML2 and K562) were injected subcutaneously into the flank of NOD/SCID mice. Four (OCI-AML2), five (MDAY-D2), or seven (K562) days after injection, once tumors were palpable, mice were then treated daily for 10 days (K562) or treated with 8 doses over 10 days (OCI-AML2) with IVM (3 mg/kg) by oral gavage in water or vehicle control (n=10 per group). MDAY-D2 mice (n=10 per group) were treated similarly but dosage escalated from 3 mg/kg (4 days) to 5 mg/kg (3 days) and 6 mg/kg (3 days) as the drug was well tolerated. Tumor volume and weight was measured over time. Compared to buffer control, oral ivermectin significantly (p<0.05) decreased tumor weight and volume in all 3 models (FIG. 2A-F) by up to 70% without any gross organ toxicity. In an OCl-AML2 xenograft, ivermectin increased apoptosis in the subcutaneous tumor as measured by Tunel staining (FIG. 2G). Of note, a dose of 3 mg/kg in mice translates to a dose of 0.24 mg/kg in humans based on scaling of body weight and surface area and appears readily achievable based on prior studies.^{9,11} Thus, the activity in the xenograft studies indicates that a therapeutic window may be achievable.

### Ivermectin Induces Intracellular Chloride Flux, Causes Increase in Cell Size and Hyperpolarization of the Plasma Membrane

As an anti-parasitic agent, IVM activates chloride channels in nematodes, causing an influx of chloride ions into the nematode's cells.¹ Thus, it was investigated whether IVM caused a similar influx of chloride ions into OCI-AML2 cells leukemia cells where IVM induced cell death after 24 hours of treatment and DU145 cells that were more resistant to IVM induced cell death (FIG. 3A). OCI-AML2 and DU145 cells were treated with 10 μM IVM for 2 hours and levels of intracellular chloride were measured by staining cells with the fluorescent dye SPQ that is quenched upon binding chloride. In OCI-AML 2 cells, IVM at concentrations that induced cell death but at times that preceded cell death, decreased SPQ fluorescence, consistent with an increase in levels of intracellular chloride (FIG. 3B). In contrast, chloride influx was not observed in DU145 cells that were resistant to 10 μM IVM (FIG. 3C).

Chloride influx can increase cell size. Therefore, changes in cell size in parallel to measuring changes in chloride flux were evaluated. As measured by flow cytometry, after 2 hours of treatment, ivermectin caused an increase in cell size in OCI-AML2 but not in the resistant DU145 cells, consistent with its effects on chloride influx (FIG. 3D, E).

In nematodes, increases in intracellular chloride after IVM treatment cause membrane hyperpolarization. Therefore, the effects of IVM on plasma and mitochondrial membrane polarization was evaluated in leukemia cells. OCI-AML2, U937, and TEX leukemia cells sensitive to ivermectin-induced death, a primary AML patient sample, DU145 and PPC-1 prostate cancer cells and primary normal hematopoietic cells were treated with increasing concentrations of ivermectin. At increasing times after incubation, plasma membrane potential was measured by staining cells with DiBAC4(3) and flow cytometric analysis. In OCI-AML2 cells, treatment with ivermectin induced membrane hyperpolarization in a dose dependent manner (FIG. 4A) and as early as after 1 hour of treatment (FIG. 4B), consistent with the influx of intracellular chloride and the effects observed in nematodes. Likewise, U937 and TEX leukemia cells as well as primary AML cells sensitive to ivermectin-induced death also demonstrated plasma membrane hyperpolarization after ivermectin treatment (FIG. 4C). In contrast, DU145 and PPC-1 cells as well as primary normal hematopoietic cells that were more resistant to ivermectin did not show changes in their plasma membrane potential when treated with up to 6 μM of ivermectin for up to 24 hours (FIG. 4D).

To determine whether the plasma membrane hyperpolarization observed after IVM treatment was related to increased chloride ion flux, the plasma membrane polarization after treating cells with IVM in buffers with and without chloride was measured. OCI-AML2 cells were treated for 5 hours with IVM in a chloride replete buffer or a chloride deplete buffer where the sodium and potassium chloride ions were replaced with equimolar gluconate salts of sodium and potassium. When added to cells in the chloride replete buffer, IVM induced plasma membrane hyperpolarization similar to cells treated in RPMI medium. However, when added to cells in chloride deplete buffer, IVM caused plasma membrane depolarization (FIG. 4E). Thus, the effects of IVM on plasma membrane polarization appear to be related to increased chloride flux.

### Ivermectin Increases Intracellular Calcium but is Not Functionally Important in Leukemia Cells

Plasma membrane hyperpolarization can lead to calcium influx²³. Therefore, the effects of IVM on calcium influx in leukemia cells was tested. OCI-AML2 cells were treated with IVM and the concentration of intracellular calcium was measured by staining cells with the ratiometric dye, Indo-1 AM. As a positive control, cells were treated with digoxin which is known to increase intracellular calcium.^{24,25} Similar to the effects of digoxin, IVM increased intracellular calcium (FIG. 4F, G). However, the increase in intracellular calcium did not appear sufficient to explain the cytotoxicity of IVM, because chelation of intra and extracellular calcium with BAPTA-AM and EDTA, respectively, did not inhibit IVM-induced cell death.

### Ivermectin Increases Intracellular Reactive Oxygen Species

Manganese chloride, cobalt chloride and mercuric chloride can lead to generation of reactive oxygen species (ROS).²⁶⁻²⁸ Therefore, IVM was tested for whether increased ROS production in leukemia cells due to the observed chloride influx. OCI-AML2 cells were treated with IVM at increasing concentrations and times of incubation. After treatment, levels of intracellular ROS were measured by staining cells with Carboxy-H₂DCFDA and flow cytometry. Treatment with IVM increased ROS production at times and concentrations that coincided with plasma membrane hyperpolarization (FIG. 5A, B). Likewise, U937 and TEX leukemia cells that were sensitive to ivermectin induced death demonstrated increased ROS generation 2 hours after ivermectin treatment (FIG. 5C). In contrast, DU145 and PPC-1 cells that were more resistant to ivermectin did not show changes in ROS generation. Likewise, primary AML cells, but not normal hematopoietic cells demonstrated increased ROS generation after ivermectin treatment (FIG. 5C).

To determine whether the increased ROS production was functionally important for IVM-induced cell death, cells were treated with IVM along with the free radical scavenger N-acetyl-L-Cysteine (NAC). NAC abrogated IVM-induced cell death, consistent with a mechanism of cell death related to ROS production and keeping with its effects on plasma membrane hyperpolarization and chloride influx (FIG. 5D).

Changes in ROS production are indicative of a biological response to ivermectin, but are very difficult to measure in the context of a clinical trial. Therefore, to identify alterations in gene expression that are a result of ROS production and could be used as biomarkers in the context of a clinical trial, gene expression profiling analysis (using Affymetrix HG U133 Plus 2.0 arrays) RNA derived from OCI-AML2 cells treated with ivermectin for 30 hr and 40 hr was undertaken. One hundred and fifty genes were deregulated >4-fold at both time points (33 under-expressed; 117 over-expressed) compared to control. Among these genes dysregulated were STAT1, which has been associated with increased ROS generation⁴⁸⁻⁵⁰ and the STAT1 downstream targets IFIT3, OAS1 and TRIM22. The upregulation of STAT1 and target genes IFIT3, OAS1 and TRIM22 after ivermectin treatment, was validated by Q-RT-PCR (FIG. 6A). Likewise, U937 and HL60 leukemia cells that were sensitive to ivermectin-induced death also demonstrated increased STAT1 mRNA. In contrast, DU145 and PPC-1 cells that were more resistant to ivermectin did not show changes in STAT1 expression (FIG. 6B). Changes in STAT1 expression in tumors from a leukemia xenograft model were also evaluated. Mice with OCI-AML2 subcutaneous xenografts were treated with ivermectin for 5 days. After treatment, tumors were harvested, mRNA extracted, and STAT1 expression measured by Q-RT PCR. STAT1 mRNA was increased in two of three tested tumors from mice treated with ivermectin compared to STAT1 mRNA expression from tumors harvested from mice treated with vehicle control (FIG. 6C). Changes in STAT1 genes were secondary to ROS production as pre-treatment with NAC blocked their upregulation (FIG. 6D), were also demonstrated.

Of note, the array dataset was compared to a ROS gene signature reported by Tothova et al²⁷. Of the 55 genes in the Tothova signature, 2/3 were expressed in our dataset. Of these 36 genes, 55% (20 genes) were found to be differentially regulated on ivermectin treatment (fold-change of 1.25 up or down, compared to the untreated control sample). Thus, ivermectin appears to induce genetic changes consistent with ROS induction.

Ivermectin Synergizes with Cytarabine and Daunorubicin

Cytarabine and daunorubicin are used in the treatment of AML and increase ROS production through mechanisms related to DNA damage (FIG. 7A, B).^{29,30} Therefore, the combination of IVM with cytarabine and/or daunorubicin was evaluated. OCI-AML2 and U937 cells were treated with increasing concentrations of IVM alone and in combination with cytarabine and/or daunorubicin. Cell growth and viability was measured 72 hours after incubation using the MTS assay. Data were analyzed by the Calcusyn median effect model where the combination index (CI) indicates synergism (CI<0.9), additively (CI=0.9-1.1) or antagonism (CI>1.1). In both OCI-AML2 and U937 leukemia cells, the combination of ivermectin and cytarabine demonstrated strong synergism with CI values at the ED₂₅, ED₅₀ and ED₇₅ of 0.51, 0.58 and 0.65, respectively in OCI AML2 cells and ED₂₅, ED₅₀ and ED₇₅ of 0.55, 0.71 and 0.91 in U937 cells (FIG. 7C). Likewise in OCI-AML2 cells, the combination of ivermectin and daunorubicin was also synergistic with CI values at the ED₂₅, ED₅₀ and ED₇₅ of 0.48, 0.51 and 0.54, respectively. The combination of ivermectin and daunorubicin, although showed some synergy at higher concentrations of IVM, lower concentrations were closer to additive in U937 with CI values at the ED₂₅, ED₅₀ and ED₇₅ of 1.1, 0.98 and 0.85, respectively (FIG. 7D The combination of ivermectin and cytarabine in normal hematopoietic cells was also tested. In contrast to the effects observed in the leukemia cell lines, ivermectin did not enhance the cytotoxicity of cytarabine in normal cells (FIG. 7E).

Drug sequencing can affect the activity of drug combinations. Therefore, the effect of drug sequencing on the synergism between ivermectin and cytarabine or daunorubicin was tested. In OCI-AML2 and U937 cells, the combination of ivermectin and cytarabine remained synergistic regardless of whether the ivermectin was given with, before or after the addition of cytarabine (FIG. 7F). In contrast, in OCI-AML2 cells, the combination of ivermectin was synergistic when given before or simultaneously with daunorubicin. However the effects of the combination were additive when the ivermectin was given after the addition of the daunorubicin (FIG. 7F). Drug sequencing may or may not have the same effect on synergy in humans.

The combination of IVM with the antihelmintic albendazole was also evaluated as this agent synergized with IVM for treatment of nematodes.^{31,32} In contrast to the synergy observed with cytarabine and daunorubicin, albendazole antagonized the anti-leukemic effects of IVM with CI values at the ED₂₅, ED₅₀ and ED₇₅ of 1.59, 1.09 and 0.89, respectively.

### Discussion

A library of drugs was screened for compounds cytotoxic to leukemia cells. From this screen the anti-parasitic agent, IVM, was identified and which induced cell death in leukemia cell lines at low micromolar concentrations and delayed tumor growth in mouse models of leukemia at doses as low as 3 mg/kg.

As part of its development as an anti-parasitic, the pharmacology and toxicology of IVM has been studied extensively in humans and animals. For example, healthy male volunteers received a 14 mg capsule of radiolabelled ivermectin. The mean Tmax was 6 hours with a half-life of 11.8 hours. IVM is metabolized in the liver, IVM and/or its metabolites are excreted almost exclusively in the feces over an estimated 12 days, with <1% of the administered dose excreted in the urine.¹⁰ In onchocerciasis patients following a single oral dose of 150 μg/kg, the maximum plasma concentration was 52.0 ng/ml, achieved in 5.2 hours with an area under the curve over 48 hours of 2852 ng.h/ml (3 μM).³³

Likewise, the toxicology of IVM in humans and animals is well described and suggests the doses of the drug required for an anti-tumor effect can be achieved in humans. For example, the LD₅₀ of oral IVM is approximately 28-30 mg/kg in mice, 80 mg/kg in dogs and above 24 mg/kg in monkeys.⁷′³³ Humans, being treated for onchocerciasis typically receive a single low dose of 100-200 μg/kg of IVM is administered as a single dose, but higher doses for longer durations have been used to treat other conditions. When used to treat onchocerciasis, hypotension, fever, adenitis, arthralgia, tachycardia and pruritis were all reported after a single dose of Ivermectin treatment.³⁴ However, these adverse effects are related to the patient's immune response (Mazzotti reaction) to dead microfilariae and not toxicity from the Ivermectin. The severity of the Mazzotti reaction is directly related to the initial intensity of infection. Thus, the doses of IVM used to treat

Onchocerciasis are much lower than the anticipated anti-tumor concentrations and may partly explain why anti-cancer effects of IVM have not been previously observed. However, doses of Ivermectin much higher than those required to treat Onchocerciasis have been administered to humans as part of the evaluation of this agent for other indications such as muscle spasticity and are within the range of doses anticipated to be required for an anti-tumor effect.⁹ Of note, in patients without onchocerciasis, Mazzotti reactions were not observed and Ivermectin was well tolerated. For example, in patients with spinal injury and resultant muscle spasticity, up to 1.6 mg/kg of Ivermectin was administered subcutaneously at twice weekly for up to 12 weeks.⁹ Finally, reports of Ivermectin overdoses also support the potential wide therapeutic window with this drug. For example, a 43 year old female in the UK with a paranoid delusional disorder self-administered 6 g of veterinary Ivermectin 30 to 50 times over the course of one year, along with furosemide and steroids. She was admitted to a London hospital for a full evaluation. Save for a Cushingoid appearance and hypokalemia, no other abnormalities were noted.¹¹ Multiple other ingestion events have also been reported, particularly in paediatric subjects who accidentally consumed of veterinary Ivermectin kept in the household for the family dog. In the majority of these, no serious adverse events were reported.^{9,11}

These studies suggest that IVM-induced cell death is related to its known function as an activator of chloride channels. As an anti-parasitic, IVM activates glutamate-gated chloride channels unique to invertebrates. However, at higher concentrations IVM can also activates mammalian chloride channels.⁷ Mammalian chloride channels broadly fall into five classes based on their regulation: cystic fibrosis transmembrane conductance regulator (CFTR), which is activated by cyclic AMP dependent phosphorylation; calcium activated chloride channels (CaCCs); voltage gated chloride channels (CICs); ligand gated chloride channels (GABA (γ-aminobutyric acid) and glycineactivated); and volume regulated chloride channels. Members of voltage gated chloride channels contain nine subtypes, CIC-1 to CIC-7, and CIC-Ka and CIC-Kb and ligand gated chloride channels act in heteromeric complexes dependent upon cell type, with multiple permutations and combinations of the subunits.³⁵ Currently, it is unclear which mammalian chloride channels are being activated by IVM.

Cells require ion channel function to maintain basic homeostatic parameters, such as intracellular Ca2+, pH and cell volume, and to allow uptake of substrates and release of metabolic products.³⁶ Strikingly, both inhibition and activation of Chloride channel activity can disrupt cellular homeostasis and impair proliferation and survival; however, the mechanisms and downstream effectors of cell death are not fully understood. Of note, malignant cells appear more sensitive to alterations in intracellular Chloride concentration, compared to untransformed cells. The basis for this therapeutic window is unclear, but without wishing to be bound to theory may relate to increased expression of Chloride channels on the surface of malignant cells, or to increased sensitivity to Chloride channel agonists and antagonists.^{37,38} Malignant cells are also more sensitive to changes in cell volume, which may also explain the observed therapeutic window.³⁹

The in vitro clonogenic studies demonstrated a narrow difference between the cytotoxicity of IVM for primary AML and normal hematopoietic cells. However, it is important to note that results of colony formation assays do not always predict clinical toxicity. For example, cytarabine and m-AMSA are chemotherapeutic agents routinely used in the treatment of AML, but show little or no selectivity for malignant cells over normal cells in colony formation assays.^{40,41} In addition, it was demonstrated that oral IVM delayed tumor growth in three mouse models of leukemia without untoward toxicity, supporting a therapeutic window. Finally, toxicology studies with IVM in animals and humans did not report hematologic toxicity.

A functional chloride channel and chloride conductance is required for beta-amyloid protein to induce generation of neurotoxic ROS in microglia cells.⁴² Furthermore, addition of cobalt chloride, manganese chloride and mercuric chloride to brain cells increases ROS production.^{27,43,44} Therefore, the effects of IVM on ROS production was examined in leukemia cells and it was demonstrated that cytotoxic concentrations of IVM increased levels of ROS. ROS generation appeared functionally important for IVM-induced death as pre-treatment with the antioxidant N-acetyl-L-cysteine (NAC) inhibited IVM-induced cell death. IVM-mediated ROS production may also explain why malignant cells are more sensitive to IVM compared to normal cells as malignant cells have higher basal levels of ROS and are less tolerant ROS-inducing agents compared to normal cells.^{45,46}

Cytarabine and daunorubicin, which are used in the treatment of AML induce ROS generation, but through a mechanism linked to DNA damage and thus a mechanism distinct from IVM. Therefore, the combination of these drugs with IVM was evaluated and IVM demonstrated synergy with both of these drugs. Therefore, IVM can be used in combination with these agents to for example enhance the efficacy of standard therapy for AML.

Accordingly, it is shown that IVM can be repurposed as a novel anti-cancer drug as it induces a cytotoxic effect in malignant cells via chloride influx, membrane hyperpolarization and increasing levels of intracellular reactive oxygen species. Given its prior safety record in humans and animals coupled with its pre-clinical efficacy in hematological malignancies demonstrated herein a phase 1 clinical trial has been designed to evaluate the tolerance and biological activity of oral IVM in patients with relapsed or refractory hematological malignancies.

### Example 2

Patients with refractory leukemia will receive increasing doses of Ivermectin daily×7 days. Next phase trial would be increasing doses of ivermectin along with standard doses of ara-C and daunorubicin chemotherapy as initial treatment for patients with high risk AML or for patients with relapsed disease.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

1. Tan K, Culjkovic B, Amri A, Borden K L. Ribavirin targets eIF4E dependent Akt survival signaling. Biochem Biophys Res Commun. 2008; 375:341-345.
2. Kentsis A, Topisirovic I, Culjkovic B, Shao L, Borden K L. Ribavirin suppresses eIF4E-mediated oncogenic transformation by physical mimicry of the 7-methyl guanosine mRNA cap. Proc Natl Acad Sci USA. 2004; 101:18105-18110.
3. Assouline S, Culjkovic B, Cocolakis E, et al. Molecular targeting of the oncogene eIF4E in acute myeloid leukemia (AML): a proof-of-principle clinical trial with ribavirin. Blood. 2009; 114:257-260.
4. Sella A, Kilbourn R, Amato R, et al. Phase II study of ketoconazole combined with weekly doxorubicin in patients with androgen-independent prostate cancer. J Clin Oncol. 1994; 12:683-688.
5. Small E J, Halabi S, Dawson N A, et al. Antiandrogen withdrawal alone or in combination with ketoconazole in androgen-independent prostate cancer patients: a phase III trial (CALGB 9583). J Clin Oncol. 2004; 22:1025-1033.
6. Mao X, Li X, Sprangers R, et al. Clioquinol inhibits the proteasome and displays preclinical activity in leukemia and myeloma. Leukemia. 2009; 23:585-590.
7. Dadarkar S S, Deore M D, Gatne M M. Comparative evaluation of acute toxicity of ivermectin by two methods after single subcutaneous administration in rats. Regul Toxicol Pharmacol. 2007; 47:257-260.
8. Brown K R, Ricci F M, Ottesen E A. Ivermectin: effectiveness in lymphatic filariasis. Parasitology. 2000; 121 Suppl:S133-146.
9. Costa J L, Diazgranados J A. Ivermectin for spasticity in spinal-cord injury. Lancet. 1994,343:739.
10. Guzzo C A, Furtek C I, Porras A G, et al. Safety, tolerability, and pharmacokinetics of escalating high doses of ivermectin in healthy adult subjects. J Clin Pharmacol. 2002; 42:1122-1133.
11. Frost M. FDA New Drug Application (NDA) for STROMECTOL (ivermectin) 6-mg for the treatment of strongyloidiasis and onchocerciasis.; 1996.
12. Simpson C D, Mawji I A, Anyiwe K, et al. Inhibition of the sodium potassium adenosine triphosphatase pump sensitizes cancer cells to anoikis and prevents distant tumor formation. Cancer Res. 2009; 69:2739-2747.
13. Mawji I A, Simpson C D, Hurren R, et al. Critical role for Fas-associated death domain-like interleukin-1-converting enzyme-like inhibitory protein in anoikis resistance and distant tumor formation. J Natl Cancer Inst. 2007; 99:811-822.
14. Buick R N, Till J E, McCulloch E A. Colony assay for proliferative blast cells circulating in myeloblastic leukaemia. Lancet. 1977; 1:862-863.
15. Eberhard Y, McDermott S, Wang X, et al. Chelation of intracellular iron with the anti-fungal agent ciclopirox olamine induces cell death in leukemia and myeloma cells. Blood. 2009.
16. Pilas B, Durack G. A flow cytometric method for measurement of intracellular chloride concentration in lymphocytes using the halide-specific probe 6-methoxy-N-(3-sulfopropyl) quinolinium (SPQ). Cytometry. 1997; 28:316-322.
17. Gurfinkel D M, Chow S, Hurren R, et al. Disruption of the endoplasmic reticulum and increases in cytoplasmic calcium are early events in cell death induced by the natural triterpenoid Asiatic acid. Apoptosis. 2006; 11:1463-1471.
18. Huang Z M, Prasad C, Britton FC, Ye L L, Hatton W J, Duan D. Functional role of CLC-2 chloride inward rectifier channels in cardiac sinoatrial nodal pacemaker cells. J Mol Cell Cardiol. 2009; 47:121-132.
19. Rothbard J B, Jessop T C, Lewis R S, Murray B A, Wender P A. Role of membrane potential and hydrogen bonding in the mechanism of translocation of guanidinium-rich peptides into cells. J Am Chem Soc. 2004; 126:9506-9507.
20. Pham N A, Jacobberger J W, Schimmer A D, Cao P, Gronda M, Hedley D W. The dietary isothiocyanate sulforaphane targets pathways of apoptosis, cell cycle arrest, and oxidative stress in human pancreatic cancer cells and inhibits tumor growth in severe combined immunodeficient mice. Mol Cancer Ther. 2004; 3:1239-1248.
21. Chou T C, Talalay P. Quantitative analysis of dose-effect relationships: the combined effects of multiple drugs or enzyme inhibitors. Adv Enzyme Regul. 1984; 22:27-55.
22. Gonzalez Canga A, Sahagun Prieto A M, Diez Liebana M J, Fernandez Martinez N, Sierra Vega M, Garcia Vieitez J J. The pharmacokinetics and interactions of ivermectin in humans—a mini-review. AAPS J. 2008; 10:42-46.
23. McCarty M F, Barroso-Aranda J, Contreras F. The hyperpolarizing impact of glycine on endothelial cells may be anti-atherogenic. Med Hypotheses. 2009; 73:263-264.
24. Meral I, Hsu W, Hembrough F B. Digoxin- and monensin-induced changes of intracellular Ca2+ concentration in isolated guinea-pig ventricular myocyte. J Vet Med A Physiol Pathol Clin Med. 2002; 49:329-333.
25. Wagner J, Bremhorst T, Schumann H J. Influence of frequency of stimulation on the toxicity of digoxin on isolated guinea-pig atria in different extracellular Ca2+. Arch Int Pharmacodyn Ther. 1978; 236:228-233.
26. Park E J, Park K. Induction of reactive oxygen species and apoptosis in BEAS-2B cells by mercuric chloride. Toxicol In Vitro. 2007; 21:789-794.
27. Zhang P, Hatter A, Liu B. Manganese chloride stimulates rat microglia to release hydrogen peroxide. Toxicol Lett. 2007; 173:88-100.
28. Kamiya T, Hara H, Yamada H, Imai H, Inagaki N, Adachi T. Cobalt chloride decreases EC-SOD expression through intracellular ROS generation and p38-MAPK pathways in COS7 cells. Free Radic Res. 2008; 42:949-956.
29. Tsang W P, Chau S P, Kong S K, Fung K P, Kwok T T. Reactive oxygen species mediate doxorubicin induced p53-independent apoptosis. Life Sci. 2003; 73:2047-2058.
30. lacobini M, Menichelli A, Palumbo G, Multari G, Werner B, Del Principe D. Involvement of oxygen radicals in cytarabine-induced apoptosis in human polymorphonuclear cells. Biochem Pharmacol. 2001; 61:1033-1040.
31. Asio S M, Simonsen P E, Onapa A W. Mansonella perstans: safety and efficacy of ivermectin alone, albendazole alone and the two drugs in combination. Ann Trop Med Parasitol. 2009; 103:31-37.
32. Demeler J, Van Zeveren A M, Kleinschmidt N, et al. Monitoring the efficacy of ivermectin and albendazole against gastro intestinal nematodes of cattle in Northern Europe. Vet Parasitol. 2009; 160:109-115.
33. Baraka O Z, Mahmoud B M, Marschke C K, Geary T G, Homeida M M, Williams J F. Ivermectin distribution in the plasma and tissues of patients infected with Onchocerca volvulus. Eur J Clin Pharmacol. 1996; 50:407-410.
34. Albiez E J, Newland H S, White A T, et al. Chemotherapy of onchocerciasis with high doses of diethylcarbamazine or a single dose of ivermectin: microfilaria levels and side effects. Trop Med Parasitol. 1988; 39:19-24.
35. Verkman A S, Galietta L J. Chloride channels as drug targets. Nat Rev Drug Discov. 2009; 8:153-171.
36. Kunzelmann K. Ion channels and cancer. J Membr Biol. 2005; 205:159-173.
37. Wang J W, Peng S Y, Li J T, et al. Identification of metastasis-associated proteins involved in gallbladder carcinoma metastasis by proteomic analysis and functional exploration of chloride intracellular channel 1. Cancer Lett. 2009; 281:71-81.
38. Abdel-Ghany M, Cheng H C, Elble R C, Pauli B U. The breast cancer beta 4 integrin and endothelial human CLCA2 mediate lung metastasis. J Biol Chem. 2001; 276:25438-25446.
39. Jiang B, Hattori N, Liu B, et al. Expression and roles of Cl-channel ClC-5 in cell cycles of myeloid cells. Biochem Biophys Res Commun. 2004; 317:192-197.
40. Singer C R, Linch D C. Comparison of the sensitivity of normal and leukaemic myeloid progenitors to in-vitro incubation with cytotoxic drugs: a study of pharmacological purging. Leuk Res. 1987; 11:953-959.
41. Spiro T E, Socquet M, Delforge A, Stryckmans P. Chemotherapeutic sensitivity of normal and leukemic hematopoietic progenitor cells to N-[4-(9-acridinylamino)-3-methoxyphenyl]-methanesulfonamide, a new anticancer agent. J Natl Cancer Inst. 1981; 66:615-618.
42. Milton R H, Abeti R, Averaimo S, et al. CLIC1 function is required for beta-amyloid-induced generation of reactive oxygen species by microglia. J Neurosci. 2008; 28:11488-11499.
43. Hussain S, Rodgers D A, Duhart H M, Ali S F. Mercuric chloride-induced reactive oxygen species and its effect on antioxidant enzymes in different regions of rat brain. J Environ Sci Health B. 1997; 32:395-409.
44. Kotake-Nara E, Saida K. Endothelin-2/vasoactive intestinal contractor: regulation of expression via reactive oxygen species induced by CoCl2, and Biological activities including neurite outgrowth in PC12 cells. Scientific World Journal. 2006; 6:176-186.
45. Kong Q, Beel J A, Lillehei K O. A threshold concept for cancer therapy. Med Hypotheses. 2000; 55:29-35.
46. Sawayama Y, Miyazaki Y, Ando K, et al. Expression of myeloperoxidase enhances the chemosensitivity of leukemia cells through the generation of reactive oxygen species and the nitration of protein. Leukemia. 2008; 22:956-964.
47. Wood T E, Dalili S, Simpson C D, et al. A novel inhibitor of glucose uptake sensitizes cells to FAS-induced cell death. Mol Cancer Ther. 2008; 7:3546-3555.
48. Kim H S, Cho I H, Kim J E, et al. Ethyl pyruvate has an anti-inflammatory effect by inhibiting ROS-dependent STAT signaling in activated microglia. Free Radic Biol Med. Oct. 1 2008; 45(7):950-963.
49. Kim H S, Lee M S. Essential role of STAT1 in caspase-independent cell death of activated macrophages through the p38 mitogen-activated protein kinase/STAT1/reactive oxygen species pathway. Mol Cell Biol. August 2005; 25(15):6821-6833.
50. Liu T, Castro S, Brasier A R, Jamaluddin M, Garofalo R P, Casola A. Reactive oxygen species mediate virus-induced STAT activation: role of tyrosine phosphatases. J Biol Chem. Jan. 23 2004; 279(4):2461-2469.

1. A method of treating a hematological malignancy comprising administering to a subject in need thereof, an effective amount of an avermectin, in combination with an effective amount of a chemotherapeutic.

2. (canceled)

3. The method of claim 1, wherein the chemotherapeutic is an anthracycline or an anthracenedione.

4. (canceled)

5. The method of claim 1, wherein the chemotherapeutic is selected from cytarabine, daunorubicin, doxorubicin, idarubicin mitoxantrone, and amsacrine, and mixtures thereof.

6. (canceled)

7. (canceled)

8. The method of claim 1 wherein the avermectin is selected from ivermectin (IVM), invermectin, avermectin, abamectin, doramectin, eprinomectin and selamectin and mixtures thereof.

9. The method of claim 1, wherein the avermectin is IVM and is administered in combination with cytarabine; or IVM administered in combination with daunorubicin.

10.-20. (canceled)

21. A method of inducing cell death in a hematological cancer cell comprising contacting the cell with an avermectin and a chemotherapeutic.

22. The method of claim 21, wherein the chemotherapeutic is cytarabine and/or daunorubicin and optionally a suitable carrier or vehicle.

23. The method of claim 21, wherein the avermectin is selected from IVM, invermectin, avermectin, abamectin, doramectin, eprinomectin and selamectin and mixtures thereof.

24. (canceled)

25. The method of claim 9, wherein IVM comprises at least 80% H2B1a and less than 20% H2B1b, optionally about 80% H2B1a and about 20% H2B1b.

26. The method of claim 1, wherein the hematological malignancy is a leukemia, myeloma or lymphoma and/or a relapsed or refractory hematological malignancy.

27. The method of claim 26 wherein the leukemia is selected from acute myeloid leukemia (AML), acute lymphocytic leukemia (ALL) and chronic myelogenous leukemia (CML).

28. The method of claim 1, wherein the avermectin and the chemotherapeutic, optionally cytarabine and/or daunorubicin are comprised in a single oral dosage form or separate oral dosage forms or in a single intravenous dosage form or separate intravenous dosage:forms.

29. (canceled)

30. A composition comprising an ᵢvermectin, in combination with a chemotherapeutic, optionally cytarabine and/or daunorubicin.

31. (canceled)

32. The composition of claim 30, wherein the avermectin is selected from ivermectin (IVM), invermectin, avermectin, abamectin, doramectin, eprinomectin and selamectin, and mixtures thereof.

33. (canceled)

34. The composition of claim 32, wherein the IVM comprises at least 80% H2B1a and less than 20% H2B1b, optionally about 80% H2B1a and about 20% H2B1b.

35. (canceled)

36. The composition of claim 30, wherein the composition is formulated as an oral dosage, optionally an oral solid dosage form or an oral liquid dosage form selected from enteric coated tablets, caplets, gelcaps, and capsules, or an injectable dosage, each unit dosage form comprising about 1 to less than about 500 mg, suitably about 1 to about 350 mg, about 1 to about 150 mg, about 1 to about 120 mg, about 1 to about 100 mg, about 1 to about 80 mg, about 1 to about 50 mg, about 1 to about 30 mg, about 5 to about 350 mg, about 5 to about 150 mg, about 5 to about 120 mg, about 5 to about 100 mg, about 5 to about 80 mg, about 5 to about 50 mg, about 5 to about 30 mg, about 3 to about 30 mg, or about 3.5 to about 5 mg, of an avermectin, and an effective amount of cytarabine and/or daunorubicin and optionally a suitable carrier or vehicle.

37.-40. (canceled)

41. A kit comprising an avermectin and instructions for administering in combination with a chemotherapeutic, optionally cytarabine and/or daunorubicin for use in a method according to claim 1.

42. A kit according to claim 41, wherein the avermectin is ivermectin.

43. A pharmaceutical pack comprising the composition of claim 30 and optionally instructions for use.
